(19) United States
(12) Patent Application Publication
(10) Pub. No.: US 2026/0224896 A1
(43) Pub. Date: Aug. 6, 2026

(54) SHORT-TERM VARIABILITY IN CARDIAC ELECTRICAL ACTIVITY TO TRIGGER EXTERNAL ACTIONS TO REDUCE ARRHYTHMIAS

(71) Applicant: Medtronic, Inc., Minneapolis, MN (US)

(72) Inventors: Michael D. Eggen, Chisago City, MN (US); Alfonso Aranda Hernandez, Shoreview, MN (US); Troy E. Jackson, Rogers, MN (US); Paul J. DeGroot, Minneapolis, MN (US); Xiaohong Zhou, Woodbury, MN (US)

(21) Appl. No.: 19/150,169

(22) PCT Filed: Jan. 22, 2024
(86) PCT No.: PCT/IB2024/050593

§ 371 Date: Jul. 22, 2025

Related U.S. Application Data
(60) Provisional application No. 63/481,843, filed on Jan. 27, 2023.

(51) Int. Cl. A61N 1/36; A61N 1/362; A61N 1/40; G16H 40/67

(52) U.S. Cl. CPC A61N 1/36114; A61N 1/36139; A61N 1/3621; A61N 1/40; G16H 40/67

(57) ABSTRACT

Predict the possible onset of a ventricular arrhythmia, and take steps to prevent the arrhythmia, which may provide improved patient outcomes and improved patient acceptance. Some examples of steps include triggering another device to deliver therapy, such as a pacemaker to output electrical stimulation therapy, a fluid delivery device to deliver a substance to reduce the likelihood of the arrhythmia or send an alert for the patient to change posture or activity. The detection techniques of this disclosure include a variety of detection algorithms. Some detection schemes may consume more resources than other detection schemes and therefore may consume more power and reduce battery life. A system implementing the techniques of this disclosure may employ a detection technique that consumes relatively lower energy at some times and at other times employ a detection technique that uses relatively higher energy.

This application claims the benefit of U.S. Provisional Patent Application Ser. No. 63/481,843, filed Jan. 27, 2023, the entire content of which is incorporated herein by reference.

### TECHNICAL FIELD

The disclosure relates medical devices and specifically to implantable cardiac rhythm medical devices.

### BACKGROUND

Cardiac rhythm management for patients may include detecting and helping patients recover from dangerous arrhythmias, such as bradycardia, pause, ventricular tachycardia (VT) or ventricular fibrillation (VF). Patients may carry medical devices, e.g., implanted, partially implanted, or wearable that may detect and monitor the cardiac activity of a patient as well as monitor other patient conditions. In some examples, such medical devices may provide therapy to help treat detected arrhythmias.

### SUMMARY

In general, the disclosure describes devices, systems, and techniques for predicting potential ventricular tachyarrhythmia, such as VT or VF, and taking action to minimize or prevent the arrhythmia. Some patients that are at risk for potentially dangerous arrhythmias may resist receiving a cardiac defibrillator, such as a wearable defibrillator or an implantable cardiac defibrillator (ICD), because of the fear of receiving electrical therapy. In some examples, antitachyarrhythmia therapy, e.g., electrical shocks, may be painful, or at least startling. The techniques of this disclosure may predict the possible onset of a ventricular arrhythmia, and take steps to prevent the arrhythmia, which may provide improved patient outcomes and improved patient acceptance.

Some examples of possible actions may include triggering another device to deliver therapy, such as a pacemaker to output electrical stimulation therapy, a fluid delivery device to deliver a drug or other substance to reduce the likelihood of the arrhythmia, or sending an alert for the patient to change posture or activity. In some examples, the arrhythmia prediction techniques of this disclosure may include a plurality of techniques, one or more of which may consume more resources than one or more others and therefore may consume more power and reduce battery life. A system implementing the techniques of this disclosure may employ an arrhythmia prediction technique that consumes relatively lower energy at some times and at other times employ an arrhythmia prediction detection technique that uses relatively higher energy.

In one example, this disclosure describes a system comprising sensing circuitry configured to sense cardiac activity of a patient; based on the sensed cardiac activity, detect a precursor to a ventricular tachyarrhythmia; and responsive to detecting the precursor to the ventricular tachyarrhythmia, deliver a non-shock therapy to inhibit an onset of the ventricular tachyarrhythmia.

In another example, this disclosure describes a method comprising sensing, by sensing circuitry of a medical system, cardiac activity of a patient; receiving, by processing circuitry of the medical system, an indication of the cardiac activity of the patient; based on the sensed cardiac activity, detecting, by the processing circuitry, that the sensed cardiac activity includes precursor to a ventricular tachyarrhythmia; and responsive to detecting the precursor to the ventricular tachyarrhythmia, causing, by the processing circuitry, the delivery of a non-shock therapy to inhibit an onset of the ventricular tachyarrhythmia.

In another example, this disclosure describes a non-transitory computer-readable storage medium comprising instructions that, when executed, cause processing circuitry of a computing device to: receive an indication of cardiac activity of a patient from sensing circuitry of a medical system, wherein: the sensing circuitry is configured to sense cardiac activity of the patient, and the computing device is a component of the medical system; based on the sensed cardiac activity, detect a precursor to a ventricular tachyarrhythmia; and responsive to detecting the precursor to the ventricular tachyarrhythmia, deliver a non-shock therapy to inhibit an onset of the ventricular tachyarrhythmia.

In another example, this disclosure describes a method comprising receiving, by processing circuitry and from sensing circuitry of a medical system configured to sense cardiac activity of a patient, an indication of the sensed cardiac activity; based on the sensed cardiac activity, detecting by the processing circuitry of the medical system, a first precursor to a ventricular tachyarrhythmia, responsive to detecting the first precursor, applying, by the processing circuitry, a precursor detection algorithm to the sensed cardiac activity; and confirming, by the processing circuitry, an indication of an onset of ventricular tachycardia based on the first precursor and the applied precursor detection algorithm; responsive to detecting the indication of the onset of ventricular tachyarrhythmia causing, by the processing circuitry, the delivery of a non-shock therapy to inhibit the onset of the ventricular tachyarrhythmia.

In another example, this disclosure describes a medical system comprising sensing circuitry configured to sense cardiac activity of a patient; processing circuitry configured to receive from the sensing circuitry an indication of the sensed cardiac activity; based on the sensed cardiac activity, detect a first precursor to a ventricular tachyarrhythmia; responsive to detecting the first precursor apply a precursor detection algorithm to the sensed cardiac activity; and confirming, by the processing circuitry, an indication of an onset of ventricular tachycardia based on the first precursor and the applied precursor detection algorithm; responsive to detecting the indication of the onset of ventricular tachyarrhythmia causing the delivery of a non-shock therapy to inhibit the onset of the ventricular tachyarrhythmia.

In another example, this disclosure describes a non-transitory computer-readable storage medium comprising instructions that, when executed, cause processing circuitry of a computing device to: receive an indication of cardiac activity of a patient from sensing circuitry of a medical system, wherein: the sensing circuitry is configured to sense cardiac activity of the patient, and the computing device is a component of the medical system; based on the sensed cardiac activity, detect a first precursor to a ventricular tachyarrhythmia; responsive to detecting the first precursor apply a precursor detection algorithm to the sensed cardiac activity; and confirm an indication of an onset of ventricular tachycardia based on the first precursor and the applied precursor detection algorithm; responsive to detecting the indication of the onset of ventricular tachyarrhythmia cause the delivery of a non-shock therapy to inhibit the onset of the ventricular tachyarrhythmia.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example system configured to predict cardiac arrhythmias according to one or more techniques of this disclosure.

FIG. 2 is a conceptual diagram illustrating an example system to predict cardiac arrhythmias including an implantable defibrillator according to one or more techniques of this disclosure.

FIG. 3 is a conceptual diagram illustrating an example system to predict cardiac arrhythmias including a wearable medical device according to one or more techniques of this disclosure.

FIG. 4 is a conceptual diagram illustrating neurological tissue of a patient according to one or more techniques of this disclosure.

FIG. 5 is a block diagram illustrating an example medical device according to one or more techniques of this disclosure.

FIG. 6 shows one example of an electrogram signal that may be sensed by one or more systems of this disclosure.

FIG. 7 is a flow chart illustrating an example operation of a system according to one or more techniques of this disclosure.

### DETAILED DESCRIPTION

The disclosure describes devices, systems, and techniques for predicting potential ventricular arrhythmia, and taking action to minimize or prevent the arrhythmia. Predicting and preventing irregular, and potentially dangerous, cardiac arrhythmias may improve patient outcomes by reducing the likelihood that the patient may enter tachyarrhythmia, such as ventricular tachycardia and ventricular fibrillation (VT/VF). The techniques of this disclosure may include devices that sense and analyze biological signals to determine whether a possible arrhythmia precursor is present.

An example technique may include analyzing short-term variability (STV). The presence of STV in a patient may be a precursor to predict an onset of VT/VF. In one example, the onset of VT/VF may be predicted in the range of one to fifteen minutes before an arrhythmia occurs. In some examples, a system may include at least two separate medical devices that may sense the cardiac rhythm, e.g., any cardiovascular implantable electronic device (CIED) such as a cardiac pacemaker, including a leadless pacemaker, an implantable cardiac monitor (ICM), an implantable cardioverter-defibrillator (ICD), or other implantable medical devices (IMD), and/or a wearable medical device and similar devices. In some examples the medical devices may independently sense and analyze the biological signals, then communicate with each other to confirm the presence of a precursor. In other examples, detecting an arrhythmia precursor by one device may trigger a second device to begin applying a precursor detection algorithm, such as the STV algorithm. When the system determines that a ventricular arrhythmia may be about to occur, one or more of the devices may deliver anti-tachyarrhythmia therapy.

In other examples, detecting one or more precursors to arrhythmia by one or more devices, such as the CIED mentioned above, hemodynamic monitors, pulse-wave velocity monitors, and similar devices, may trigger neurocardiologic therapies to restore the STV parameters and prevent an imminent tachyarrhythmia. For example, when STV parameter reaches given threshold, a neurostimulator, such as an implantable neurostimulator (INS), may initiate electrical stimulation therapy to neural tissue of the patient, e.g., to reduce STV and prevent a life threatening arrhythmia. Some examples may include vagal nerve stimulation, left stellate ganglion block or other neural tissue stimulation.

In other examples, one or more devices may deliver refractory period stimulation. Refractory period stimulation may different that applying stimuli in the excitable (non-refractory) period. For example, unlike non-refractory period stimulation delivered to cardiac tissue, which may be intended to the capture cardiac tissue, electrical stimulation delivered during a refractory period may be non-capturing stimulus intended to modify the refractory with a goal of preventing VT/VF onset. In this disclosure, “capture” cardiac tissue means activate the myocardium. For example, a capture threshold of an electrical stimulus may refer to a measurement of the minimal voltage or minimal current capture threshold is the minimum current setting to produce a depolarisation of cardiac tissue, e.g., of the heart chamber to which the electrical stimulus was applied.

In other examples, detecting one or more precursors to arrhythmia may trigger an electronic message to an external computing device, such as a smart phone, to notify the patient, and/or a caregiver that the patient should take some action. Some example notifications may include instructions to take an antiarrhythmic, discontinue caffeine intake, change posture or activity or other similar actions.

In other examples, a first arrhythmia prediction, e.g., precursor detection, algorithm may trigger a second precursor detection algorithm, where the second precursor detection algorithm consumes more resources than the first precursor detection algorithm. Some examples of resources may include battery power consumption, processor time, memory storage, and similar resources of the medical device(s). Any of the actions described above may be considered anti-tachyarrhythmia therapy.

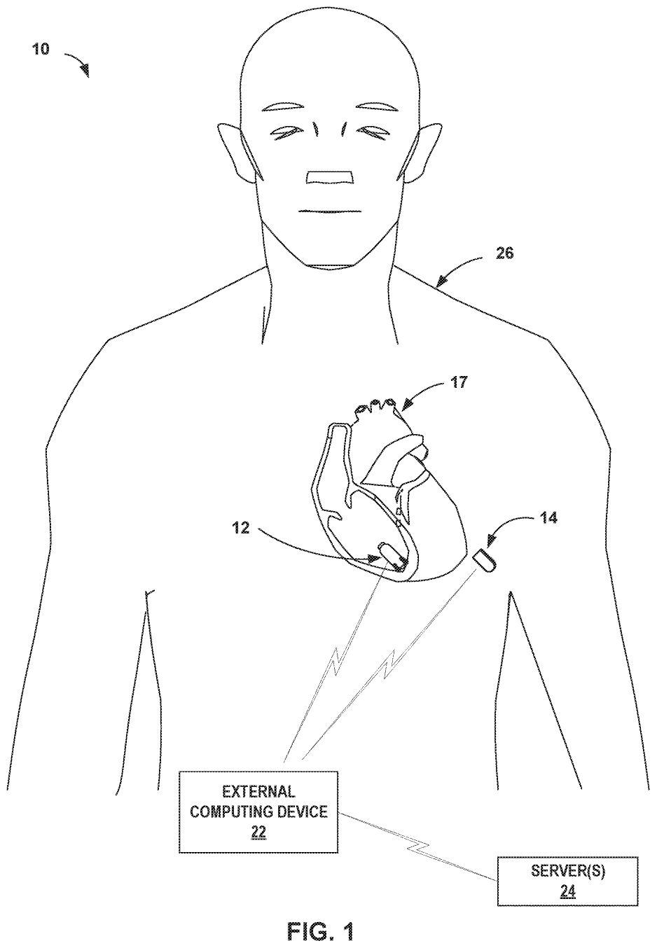

FIG. 1 is a conceptual diagram illustrating an example medical device system 10 system configured to predict cardiac arrhythmias according to one or more techniques of this disclosure. The systems, devices, and methods described in this disclosure may include example configurations of medical device system 10 having one or more implantable medical device, such as IMD 12 and IMD 14 implanted or partially implanted within a patient. For purposes of this description, knowledge of cardiovascular anatomy and functionality is presumed, and details are omitted except to the extent necessary or desirable to explain the context of the techniques of this disclosure.

System 10 includes IMD 14 and IMD 12, implanted at or near the site of a heart 17 of a patient 26, an external computing device 22, and one or more servers 24.

IMD 14 and IMD 12 may be in wireless communication with at least one of external computing device 22, servers 24, and other devices not pictured in FIG. 1.

In some examples, IMD 14, or similar medical device, may implanted outside of a thoracic cavity of patient 26 (e.g., subcutaneously in the pectoral location illustrated in FIG. 1). In other examples, IMD 14 may be positioned near the sternum near or just below the level of the heart of patient 26, e.g., at least partially within the cardiac silhouette. In other examples, IMD 14 may be implanted proximate to, attached to, or on the epicardium of heart 17. In other examples, IMD 14 may be located in other locations on patient 26, including for monitoring and stimulation of the tibial nerve, subcutaneous nerves, sacral nerve, spinal cord, vagal nerve, deep brain stimulation, located at or near one or more organs or other locations. In some examples, IMD 14 may be implemented as an insertable cardiac monitor (ICM), for long-term (chronic) monitoring of cardiac activity of patient 26.

IMD 14 includes a plurality of electrodes and may be configured to sense a cardiac electrogram (EGM) and other bioelectrical signals via the plurality of electrodes. In some examples, electrodes may be integrated with the housing of IMD 14. In various examples, IMD 14 may represent a cardiac monitor, a defibrillator, a cardiac resynchronization pacemaker or defibrillator, a pacemaker, an implantable pressure sensor, a neurostimulator, glucose monitor, drug pump, pulse wave velocity measurement device or any other implantable or external medical device.

In some examples, IMD 12 may be described as pacing device 12. Pacing device 12 may be, for example, an implantable leadless pacing device that is configured for implantation entirely within one of the chambers of heart 17, and that provides electrical signals to heart 17 via electrodes carried on the housing of pacing device 12, aka IMD 12. IMD 12 may configured to be implanted proximal to the heart of patient 26, e.g., to monitor electrical activity of the heart and/or provide electrical therapy to the heart. In some examples IMD 251 may be implemented as a ventricular-from-atrial (VfA) cardiac device and may be implanted in the right atrium (RA) with an electrode extending from the right atrium into the left ventricular (LV) myocardium.

In some examples, IMD 12 may be attached within a chamber of heart 17 as an intracardiac pacing device. In other examples that are consistent with aspects of this disclosure, IMD 12 may be attached to an external surface of heart 17, such that IMD 12 is disposed outside of heart 17 but can pace a desired chamber. In one example, IMD 12 is attached to an external surface of heart 17, and one or more components of IMD 12 may be in contact with the epicardium of heart 17. In the example of FIG. 1, IMD 12 is schematically shown attached to a wall of a ventricle of heart 17 via one or more fixation elements (e.g. tines, helix, etc.) that penetrate the tissue. These fixation elements may secure IMD 12 to the cardiac tissue and retain an electrode (e.g., a cathode or an anode) in contact with the cardiac tissue. IMD 12 may be implanted at or proximate to the apex of the heart. In other examples, a pacing device may be implanted at other ventricular locations, e.g., on the free-wall or septum, an atrial location, any location on or within heart 17 or other locations within the body of patient 26, as described above for IMD 14. For example, though the example of FIG. 1 depicts IMD 12 and IMD 14 as leadless IMDs, in other examples, the techniques of this disclosure may equally apply to transvenous IMDs. In other words, IMD 14, or a third IMD not shown in FIG. 1, may be implanted in a pocket in the pectoral region of patient 26 with a lead running from the IMD to one or more locations in or on heart 17. As another examples, IMD 12 may be implanted, or coupled to leads implanted in the anterior mediastinum or another extracardiac location.

IMD 12 may include a housing that is hermetically or near-hermetically sealed to help prevent fluid ingress into the housing. IMD 12 may include electronic components e.g., sensing circuitry for sensing cardiac electrical activity via electrodes and therapy generation circuitry for delivering electrical stimulation therapy via the electrodes.

Electronic components may include any discrete and/or integrated electronic circuit components that implement analog and/or digital circuits capable of producing the functions attributed to pacing device 12 described herein. In some examples, IMD 12 may also include components for sensing other physiological parameters, such as acceleration, pressure, sound, and/or impedance.

As with IMD 14, IMD 12 may include a memory that includes instructions that, when executed by processing circuitry within the housing of IMD 12, cause components of IMD 12 to perform various functions attributed to IMD 12. In some examples, the housing may also house communication circuitry that enables IMD 12 to communicate with other electronic devices, such as external computing device 22, which may be a medical device programmer, patient monitor or other external device. In some examples, IMD 12 may also include a power source, such as a battery.

In some examples, external computing device 22 may be a computing device with a display viewable by the user and an interface for providing input to external computing device 22 (i.e., a user input mechanism). In some examples, external computing device 22 may be a notebook computer, tablet computer, workstation, one or more servers, cellular phone, personal digital assistant, patient monitor or another computing device that may run an application that enables the computing device to interact with IMDs 12 and 14. External computing device 22 is configured to communicate with IMDs 12 and 14 and, optionally, other device (not illustrated in FIG. 1), and one or more servers 24, e.g., via wireless communication. External computing device 22, for example, may communicate via near-field communication technologies (e.g., inductive coupling, NFC, or other communication technologies operable at ranges less than 10-20 cm), far-field communication technologies (e.g., RF telemetry according to the 802.11 or Bluetooth® specification sets, or other communication technologies operable at ranges greater than near-field communication technologies), or wired communication (e.g., Ethernet).

External computing device 22 may be used to retrieve data collected by IMD 12 and IMD 14 and configure operational parameters for IMD 12 and IMD 14. To simplify the explanation, the remaining description may refer only to IMD 14, but the same description applies to IMD 12, unless otherwise noted.

The retrieved data may include values of physiological parameters measured by IMD 14, indications of episodes of arrhythmia or other maladies detected by IMD 14, and physiological signals recorded by IMD 14. For example, external computing device 22 may retrieve cardiac EGM segments recorded by IMD 14, e.g., due to IMD 14 determining that an episode of arrhythmia or another malady occurred during the segment, or in response to a request to record the segment from patient 26 or another user. In some examples, one or more remote computing devices may interact with IMD 14 in a manner similar to external computing device 22, e.g., to program IMD 14 and/or retrieve data from IMD 14, via a network.

In various examples, IMD 14 may include one or more additional sensor circuits configured to sense a particular physiological or neurological parameter associated with patient 26. For example, IMD 14 may include a sensor operable to sense a body temperature of patient 26 in a location of the IMD 14, or at the location of the patient where a temperature sensor coupled by a lead to IMD 14 is located (not shown in FIG. 1). In another example, IMD 14 may include a sensor configured to sense motion or position, e.g., and accelerometer, to sense steps taken by patient 26 and/or a position or a change of posture of patient 26. In various examples, IMD 14 may include a sensor that is configured to detect breaths taken by patient 26. In various examples, IMD 14 may include a sensor configured to detect heartbeats of patient 26. In various examples, IMD 14 may include a sensor that is configured to measure systemic blood pressure of patient 26 or other biological measurements.

In some examples, system 10 may include one or more other sensors (not shown in FIG. 1) implanted within patient 26, that is, implanted below at least the skin level of the patient. In some examples, one or more of the sensors of IMD 14 may be located externally to patient 26, for example as part of a cuff or as a wearable device, such as a device embedded in clothing that is worn by patient 26. In various examples, IMD 14 may be configured to sense one or more physiological parameters associated with patient 26, and to transmit data corresponding to the sensed physiological parameter or parameters to external computing device 22.

Transmission of data from IMD 14 to external computing device 22 in various examples may be performed via wireless transmission, using for example any of the formats for wireless communication described above. In various examples, IMD 14 may communicate wirelessly to an external device (e.g., an instrument or instruments) other than or in addition to external computing device 22, such as a transceiver or an access point that provides a wireless communication link between IMD 14 and a network.

Examples of communication techniques used by any of the devices described above with respect to FIG. 1 may include radiofrequency (RF) telemetry, which may be an RF link established via Bluetooth®, Wi-Fi, or medical implant communication service (MICS).

In some examples, system 10 may include more or fewer components than depicted in FIG. 1. For example, in some examples, system 10 may include multiple additional IMDs, such as implantable cardiac devices or other IMDs, implanted within patient 26. As another example, system 10 may include only a single IMD, such as only IMD 14.

For the single IMD example, IMD 14 may perform some analysis of the cardiac activity of patient 26 where the analysis consumes a first level of resources. The first analysis at the first level of resources may trigger processing circuitry of IMD 14 to perform a second analysis of the cardiac activity of patient 26 that consumes a second level of resources, where the second level of resources is greater than the first level of resources. For example, the second analysis may take longer than the first analysis and/or consume more energy from the battery of IMD 14 than the first analysis. Based on the first analysis and the second analysis, IMD 14 may deliver non-shock anti-arrhythmia therapy to patient 26.

In some examples, IMD 14 may start the first analysis based on a triggering event. The triggering event may include any one or more of the expiration of a time period, e.g., an hourly, daily, weekly or some other periodic analysis, or the triggering event may be based on the cardiac activity. For example, IMD 14 may detect one or more pre-ventricular contractions (PVC), a brief interval of tachycardia, one or more intervals of the cardiac cycle that satisfy a threshold, or some other triggering cardiac activity.

In other examples, as described above, the first analysis from IMD 14 may trigger IMD 12 to perform the second analysis. Based on the first analysis and the second analysis indicating a precursor to an arrhythmia, either or both of IMD 12 or IMD 14 may deliver non-shock anti-arrhythmia therapy to patient 26. A second analysis may be any combination of the same analysis repeated from a different device in a different location, e.g., with different electrodes receiving cardiac signals through a different pathway, the same or different device repeating the same analysis more frequently or for a longer duration, a different analysis of bioelectrical signals with the same or different device and/or processing circuitry e.g., an STV analysis as described herein, or any similar technique that may confirm a precursor to tachyarrhythmia.

For the remainder of the disclosure, a general reference to a medical device system may refer collectively to include any examples of medical device system 10, a general reference to IMD 14 may refer collectively to include any examples of IMD 14 and IMD 12, a general reference to sensor circuits may refer collectively to include any examples of sensor circuits of IMD 14 and IMD 12, and a general reference to an external device may refer collectively to any examples of external computing device 22.

In one example of operation of system 10, the combination of IMD 14 with IMD 12 may capture dispersion of electrical signals in a wider heart region than either IMD 12 or IMD 14 alone. In other words, system 10 may measure cardiac activity using both IMD 12 and IMD 14, and analyze the combined results to trigger therapy, or avoid triggering unnecessary therapy. For example, as described above, one or both of IMD 12 and IMD 14 may analyze a sensed cardiac rhythm using a short-term variability algorithm. As noted above, the presence of STV in a patient may be a precursor to predict an onset of a tachyarrhythmia. In some examples, external computing device 22, and or servers 24 may also analyze the sensed cardiac rhythm. Should any of the processing circuitry of any component of system 10 detect STV in the cardiac signals satisfying one or more criteria, or some other tachyarrhythmia precursor, the processing circuitry may trigger anti-tachyarrhythmia therapy. Examples of anti-tachyarrhythmia therapy may include overdrive pacing, or other high rate pacing, e.g., delivered by IMD 12, communication with a pump (not shown in FIG. 1) to automatically infuse a substance, trigger a neurostimulator to output neurocardiologic therapies to restore the STV parameters and prevent an imminent tachyarrhythmia, or some other shock or non-shock anti-tachyarrhythmia therapy as described above.

In this disclosure, STV may be measured in many different ways. In some examples, STV may be measured in time (temporal dispersion), such as variability in portions of the cardiac cycle, including Q-T variability, S-T variability, which may also be referred to as variability in the activation recovery interval (ARI), P-P interval variability and other temporal measurements of the cardiac cycle. In other examples, STV may also describe variability in amplitude (e.g., variation in T-wave maximum and kind of T-wave alternans), as well as variability in morphology.

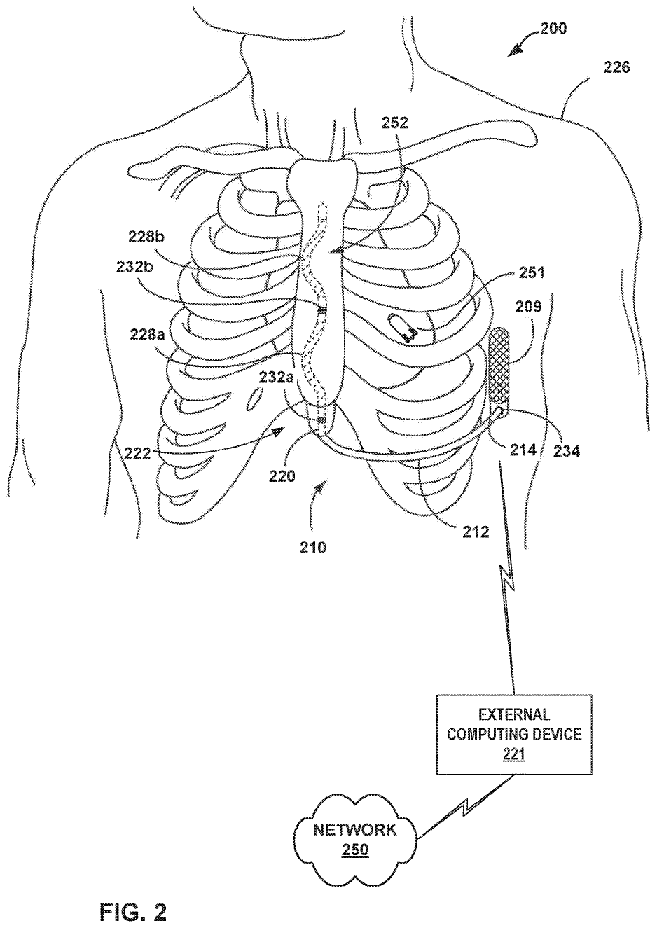

FIG. 2 is a conceptual diagram illustrating an example system to predict cardiac arrhythmias including an implantable cardioverter-defibrillator (ICD) according to one or more techniques of this disclosure. The example of FIG. 2 shows a front view of patient 226 implanted with the extra-cardiovascular ICD system implanted intra-thoracically as well as one or more other IMDs, e.g., IMD 251. The extra-cardiovascular ICD may also be referred to as an extravascular implantable cardioverter defibrillator (EV-ICD) in this disclosure. Similar to system 10, described above in relation to FIG. 1, system 200 may include one or more external computing devices 221 configured to communicate with EV-ICD 209 and IMD 251, as well as to one or more servers (not shown in FIG. 2) via network 250. EV-ICD 209 and IMD 251 may also be configured to communicate directly with each other, as described above in relation to FIG. 1.

ICD system 210 includes an EV-ICD 209 connected to a medical electrical lead 212. EV-ICD 209 may include a housing that forms a hermetic seal that protects components of the EV-ICD 209. The housing of EV-ICD 209 may be formed of a conductive material, such as titanium or titanium alloy, which may function as a housing electrode (sometimes referred to as a can electrode). In other embodiments, EV-ICD 209 may be formed to have or may include one or more electrodes on the outermost portion of the housing. EV-ICD 209 may also include a connector assembly (also referred to as a connector block or header) that includes electrical feedthroughs through which electrical connections are made between conductors of lead 212 and electronic components included within the housing of EV-ICD 209. The housing may enclose one or more processors, memories, transmitters, receivers, sensors, sensing circuitry, therapy circuitry, power sources and other appropriate components. The housing is configured to be implanted in a patient, such as the patient.

In the example of FIG. 2, EV-ICD 209 is implanted extra-thoracically on the left side of the patient, e.g., under the skin and outside the ribcage (subcutaneously or submuscularly). EV-ICD 209 may, in some instances, be implanted between the left posterior axillary line and the left anterior axillary line of the patient. EV-ICD 209 may, however, be implanted at other extra-thoracic locations on the patient as described later and, in some instances, may be implanted intra-thoracically.

In some examples, at least a portion of lead 212 may be implanted intrathoracically, e.g., between sternum 252 and the heart as shown in FIG. 2. In other examples, lead 212 may be implanted extra-thoracically, e.g., under the skin, but outside the ribs, or in other locations, depending on the anatomy and condition of the patient. In still other example, lead 212 may be implanted within the heart (e.g., intracardiac or transvenous) or attached to the heart (e.g., epicardial or pericardial). Lead 212 may include proximal end 214 with one or more connectors 234 to electrically couple the lead 212 to EV-ICD 209. In some examples, at least a part of distal portion 222 may define an undulating configuration distal to proximal end 214. Lead 212 may include defibrillation electrode segments 228a and 228b (collectively segments 228) spaced a distance apart from each other along the length of the distal portion 222. Though FIG. 2 depicts two such segments 228, in other examples, lead 212 may include one or more segments 228.

Segments 228 may function as separate defibrillation electrodes in some examples. Each segment 228 may have its own separate conductor such that a voltage may be applied to each electrode independently, or simultaneously.

Defibrillation electrode segments 228 may be a disposed around or within the lead body 212 of the distal portion 222, or alternatively, may be embedded within the wall of the lead body 212. In one configuration, the defibrillation electrode segments 228 may be a coil electrode formed by a conductor. The conductor may be formed of one or more conductive polymers, ceramics, metal-polymer composites, semiconductors, metals, or metal alloys, including but not limited to, one of or a combination of the platinum, tantalum, titanium, niobium, zirconium, ruthenium, indium, gold, palladium, iron, zinc, silver, nickel, aluminum, molybdenum, stainless steel, MP35N, carbon, copper, polyaniline, polypyrrole and other polymers. In another configuration, each of the defibrillation electrodes segments 228 may be a flat ribbon electrode, a paddle electrode, a braided or woven electrode, a mesh electrode, a directional electrode, a patch electrode, or another type of electrode configured to deliver a cardioversion/defibrillation shock to the patient's heart.

Distal portion 222 may define one or more gaps 220 between adjacent defibrillation segments 228. Gaps 220 may define any length. In instances in which more than two defibrillation segments 228 exist, each gap 220 may define the same or substantially the same length as every other gap 220 or may define a different length than other gap 220 in the distal portion. In some examples, one or more electrodes 232 may be disposed within a respective gap 220. In the configuration shown in FIG. 2, a single electrode 232a is disposed within gap 220. However, in other examples, more than one electrode 232 may exist within each respective gap 220. In the configuration shown in FIG. 2, another electrode 232b is located distal to defibrillation electrode segment 228a.

In other configurations, additional electrodes 232 may be disposed along the distal portion 222 of lead 212, e.g., distal to defibrillation electrode segment 228b and/or proximal to electrode segment 228a.

Electrodes 232a and 232b may be configured to deliver low-voltage electrical pulses to the heart or may sense a cardiac electrical activity, e.g., depolarization and repolarization of the heart. As such, electrodes 232 may be referred to herein as pace/sense electrodes 232. In one configuration, electrodes 232 are ring electrodes.

However, in other configurations the electrodes 232 may be any of a number of different types of electrodes, including ring electrodes, short coil electrodes, paddle electrodes, hemispherical electrodes, directional electrodes, or the like. Electrodes 232 may respectively be the same or different types of electrodes. Electrodes 232 may be electrically isolated from an adjacent defibrillation segment 228 by including an electrically insulating layer of material between the electrodes 232 and the adjacent defibrillation segments 228. Similar to segments 228 described above, each electrode 232 may have its own separate conductor such that a voltage may be applied to each electrode independently from another electrode 232, and from segments 28, in lead 212. In other configurations, each electrode 232 may be coupled to a common conductor such that each electrode 232 may apply a voltage simultaneously.

IMD 251 is an example of IMD 12 or IMD 14 described above in relation to FIG. 1 and may have the same functions and characteristics. In some examples, IMD 251 may be any other CIED described above, such as a neurostimulator, drug pump, pulse-wave velocity detector or similar medical device. In some examples IMD 251 may be configured to measure cardiac activity. In some examples system 200 may measure arrhythmia precursors, such STV described above, based on one or both an EV-ICD 209 vector and an electrogram (EGM) from IMD 251. In some instances, measuring arrhythmia precursors using a combined measurement may provide increased sensitivity. Should system 200 detect an arrhythmia precursor, then IMD 251 may be configured to deliver anti-arrhythmia therapy, such as overdrive pacing reduce the STV, or other precursor and avoid VT/VF. In some examples, the detected precursor may also trigger IMD 251 to apply conduction system anti-tachycardia pacing (ATP).

In some examples, EV-ICD 209 may apply the anti-arrhythmia therapy. Some patients may perceive pacing with the EV-ICD lead, e.g., overdrive pacing delivered by electrodes 232. Pacing thresholds via electrodes 232 may be relatively high compared to intracardiac leads, or a leadless device, such as IMD 251. Therefore, in some examples, overdrive pacing or other high rate pacing by IMD 251 may be preferred over such pacing from EV-ICD 209.

Overdrive pacing may differ from anti-tachycardia pacing in that overdrive pacing may be lower energy and less perceptible to the patient. Overdrive pacing may help prevent a tachyarrhythmia, where ATP may be used to pace patient 226 out of an ongoing tachyarrhythmia event. Overdrive pacing may be described in some examples as system 200 may respond to variations in the cardiac rate, or other sensed cardiac activity, by accelerating the pacing rate until the pacing rate reaches a stable paced rhythm that is slightly faster than the spontaneous rate. After each sensed, non-refractory cardiac event, the pacing device of system 200, e.g., IMD 251 or IMD 209, may shorten the cardiac pacing interval by a programmed decremental value. If the next cardiac event is another non-refractory sensed event, the pacing interval is further decremented. This overdriving function may continue until the pacing rate surpasses the spontaneous rate, resulting in a paced cardiac rhythm. After a programmable period of cardiac pacing at 100%, the pacing device may gradually decrease the pacing rate, like a smoothing function, in search of the next spontaneous sinus cycle. High rate pacing may differ from overdrive pacing in that high rate pacing may be a pre-set pacing rate rather than an accelerating pacing rate that paces faster until the pacing rate is stable, as in overdrive pacing.

In some examples, EV-ICD 209 or IMD 251, as with any device in this disclosure, may also adjust the pacing parameters during overdrive pacing to increase the pacing energy, such as increase the pacing amplitude, pulse width or some combination of pacing parameters to output a higher energy pacing pulse. Early recurrences within about ten minutes after termination of a tachyarrhythmia, such as atrial or ventricular tachycardia, flutter, or fibrillation episodes, may account for some paroxysmal tachyarrhythmia episodes in some patients. In some examples, system 200 may be configured to deliver overdrive pacing following a tachyarrhythmia episode termination.

In some examples, EV-ICD 209 or IMD 251 of system 200 may measure PVC burden as an arrhythmia precursor. As described above in relation to FIG. 1, system 10 of FIG. 1, or any other example in this disclosure may also perform the same or similar functions. In other examples, PVC burden may trigger other algorithms, such as a short-term variability algorithm for VT/VF prediction. PVC burden may be defined as the percentage or other amount PVCs of the total number of QRS complexes during a time period, e.g., 24 hours. In case of multiple PVC morphologies, the bundle branch block and QRS duration of the dominant morphology may be used for analysis. Among patients presenting for clinical care for their heart failure and PVCs, a higher burden of PVCs may indicate a risk factor. Patients who have a high PVC burden, e.g., more than ten percent of total heartbeats may benefit from an evaluation of their systolic function. Patients with a very high PVC burden, e.g., more than 20 percent of total heartbeats may have an increased risk of arrhythmia-induced cardiomyopathy. In some examples, one or more components of system 200 may evaluate the cardiac activity of patient 226 for PVC burden and trigger an action based on one or more thresholds. For example, a first threshold may trigger an STV algorithm, or some other algorithm to confirm that the patient may be at risk for an imminent arrhythmia. A second threshold, e.g., a higher percentage than the first threshold, may trigger one or more anti-tachyarrhythmia therapies described above, such as overdrive pacing.

In this disclosure, an “imminent” onset of an arrhythmia means the possibility of an arrhythmia occurring within a time frame of seconds, minutes or within hours of detecting an indication of the onset of an arrhythmia. In some examples, and depending on the patient, the particular indication and other factors, processing circuitry of the systems of this disclosure may automatically take different actions in response to detecting the indication. As described above, actions may range from a confirmation of the indication to application of a non-shock anti-tachyarrhythmia therapy.

In some examples, an STV algorithm of this disclosure may include the determination as to whether the STV metric satisfies one or more therapy delivery thresholds. In addition, or alternatively, the STV algorithm may include identifying one or more trends in the STV metric over one or more days or weeks and/or one or more comparison(s) of the STV metric to the STV metric(s) determined for previous days. For example, one or more patient-specific therapy delivery thresholds may be determined based on an increase (e.g., fixed value or percentage) over a programmed patient's baseline STV value(s) over the course of one or more days. As another example, one or more patient-specific therapy delivery thresholds may be determined based on a trending increase (e.g., fixed value or percentage) of the patient's STV value(s) over one or more prior days.

As another example, one or more patient-specific therapy delivery thresholds may be automatically programmed after an arrhythmic event is detected by the device. In such examples, the device may detect an arrhythmic event which was not predicted ahead of time, and then automatically adjust and re-program the STV threshold for the patient based on the patient's STV and the detected arrhythmic event. In addition, the determining as to whether the STV metric satisfies one or more therapy delivery thresholds may also be based on a patient population to which the patient belongs. For example, the therapy delivery thresholds may be based on different patient populations, such as age, gender, amount of scar tissue, comorbidities, etc.) and the system may determine the patient-specific therapy delivery thresholds based on the patient's membership in one or more patient populations.

In some examples, system 200 may measure the number, frequency, or some similar metric of non-sustained ventricular tachycardia (NSVT) as a precursor to tachyarrhythmia in patient 226. A metric of non-sustained arrhythmias within an individual patient may be used for predicting the occurrence of a sustained arrhythmia.

Variables of interest for such a metric may include, but are not limited to, the number of non-sustained arrhythmias occurring during a specified period of time, the duration of the non-sustained arrhythmias, the atrial and/or ventricular intervals during the non-sustained arrhythmias, and characteristics of the EGM morphology during non-sustained arrhythmias. One or more of these variables may be used to determine one or more non-sustained arrhythmia metrics. The metric(s) may then be used in other device operations, such as a precursor to deliver one or more anti-tachyarrhythmia therapies described above.

In some examples, the metric for NSVT may reflect changes in the frequency or duration of non-sustained arrhythmias, which may indicate changes in factors responsible for triggering a sustained arrhythmia. Some additional examples of such metrics may include the frequency of non-sustained arrhythmia episodes determined as the number of episodes occurring within a predetermined amount of time. Another metric may be the average duration of a given number of non-sustained episodes or the average of a number, such as 20, 30, or some other number of all non-sustained episodes occurring within a predetermined amount of time. Another example metric may include an NSVT index defined as the product of the number of NSVT episodes/day times the mean number of beats per episode, i.e., total NSVT beats per day (total beats/day), and may represent a severity of NSVT incidences. Other example metrics may be equal to the value of the episode counter and represent the frequency of non-sustained arrhythmia episodes during one timer cycle. Another metric may be calculated as the average of the stored episode durations or the total number of non-sustained arrhythmia intervals occurring during a timer interval calculated as the sum of all the stored episode durations. A metric may be the product of the number of episodes detected and the average of all episode durations.

Processing circuitry of system 200 may determine one or more non-sustained arrhythmia metrics based on the stored cycle interval(s) and/or the EGM morphology template data. A non-sustained arrhythmia metric may be calculated as an average of cycle interval data collected or an average of a characteristic feature of the EGM template. Processing circuitry of system 200 may use any such metrics, or combination of metrics as a precursor to an arrhythmia and trigger further action, such as additional analysis by another algorithm, or by delivering any of the anti-tachyarrhythmia therapies described above.

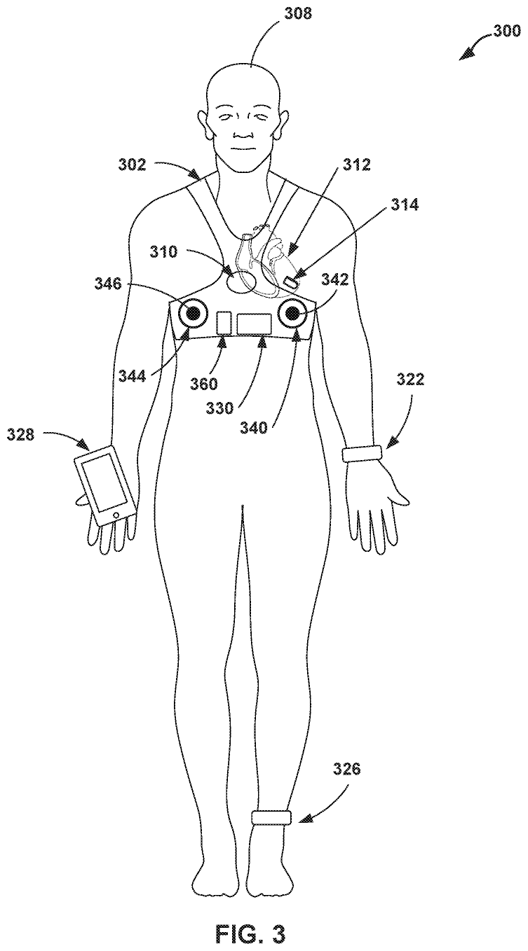

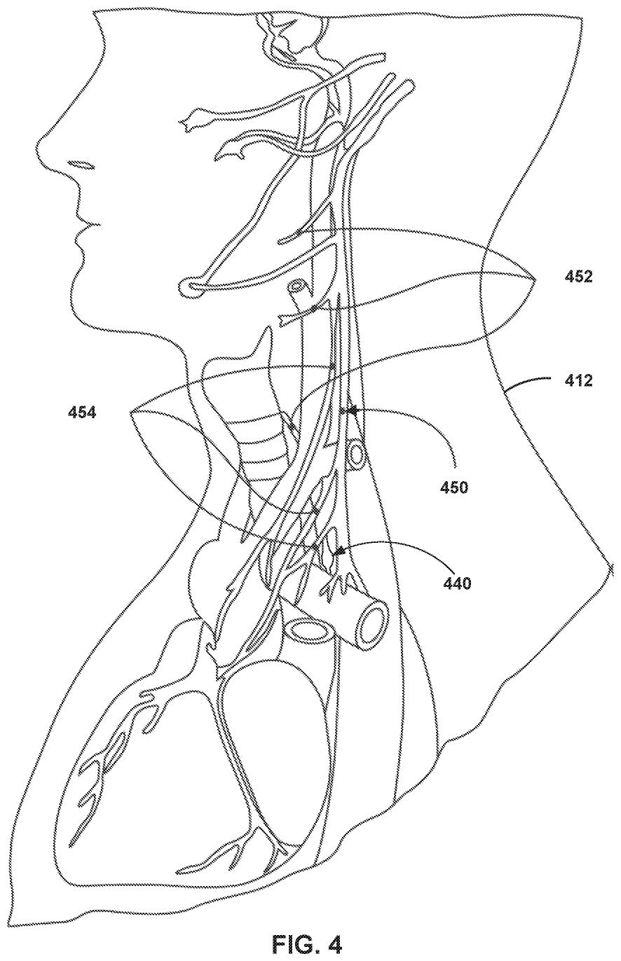

FIG. 3 is a conceptual diagram illustrating an example system to predict cardiac arrhythmias including a wearable medical device according to one or more techniques of this disclosure. System 300 in the example of FIG. 3 is another example of system 10 and system 200 described above in relation to FIGS. 1 and 2 respectively. In the example of FIG. 3, system 300 includes wearable garment 302, one or more other wearable devices 322 and 326, a portable computing device 328 and one or more implantable or other wearable devices, such as IMD 314. Similar to system 10 and 200, system 300 may include an external computing device, in addition to portable computing device 328, and any of wearable garment 302, wearable devices 322 and 326 and a portable computing device 328 may communicate via a network (not shown in FIG. 3).

IMD 314 may perform any of the functions and have the same or similar characteristics to IMD 12, IMD 14, EV-ICD 209 and IMD 251 described above in relation to FIGS. 1 and 2.

Wearable garment 302 is a medical device that, in the example of FIG. 3, includes a plurality of sensing electrodes 340, 342, 344, 346 located on the garment such that the plurality of sensing electrodes receives bioelectrical signals from the skin of patient 308. Garment 302 also includes processing circuitry 310 and therapy delivery electrode 330. Garment 302 may also include other therapy delivery electrodes, e.g., along the side or back of patient 308 (not shown in FIG. 3). In some examples, garment 302 may include a mechanical sensor configured to output an indication of a compression level of garment 302 and an apparatus configured to adjust the compression level of the garment (not shown in FIG. 3). Power source 360 may provide power to the components of wearable garment 302. In some examples power source 360 may be a replaceable or rechargeable battery. In some examples, power source 360 may be mounted on garment 302. In other examples, power source 360 may be carried externally and connected to garment 302, e.g., carried in a separate belt pack, a purse, and so on.

Processing circuitry 310 may be attached to garment 302 and operatively coupled to the mechanical sensor, the sensing electrodes 340, 342, 344 and 346, and the apparatus for adjusting the compression level of garment 302. Processing circuitry 310 may be an example of a programmable processor, which may include any one or more of a microcontroller (MCU), e.g. a computer on a single integrated circuit containing a processor core, memory, and programmable input/output peripherals, a microprocessor (uP), e.g. a central processing unit (CPU) on a single integrated circuit (IC), a controller, a digital signal processor (DSP), an application specific integrated circuit (ASIC), a field-programmable gate array (FPGA), a system on chip (SoC) or equivalent discrete or integrated logic circuitry. A programmable processor may be integrated circuitry, i.e., integrated processing circuitry, and that the integrated processing circuitry may be realized as fixed hardware processing circuitry, programmable processing circuitry and/or a combination of both fixed and programmable processing circuitry. Accordingly, the terms “processing circuitry,” “processor” or “controller,” as used herein, may refer to any one or more of the foregoing structures or any other structure operable to perform techniques described herein.

Examples of a memory may include any type of computer-readable storage media, such as random access memory (RAM), read only memory (ROM), programmable read only memory (PROM), erasable programmable read only memory (EPROM), electronically erasable programmable read only memory (EEPROM), flash memory, and similar devices. In some examples the computer readable storage media may store instructions that cause the processing circuitry to execute the functions described herein.

In some examples, the computer readable storage media may store data, such as configuration information, temporary values and other types of data used to perform the functions of this disclosure.

In the example of FIG. 3, therapy electrodes 330 and sensing electrodes 340, 342, 344 and 346 are configured to be placed in contact with skin of a patient and held in position with a compressive member, such as garment 302. Both therapy electrodes 330 and sensing electrodes 340, 342, 344 and 346 may be free of adhesives. Electrodes held in position only with the compressive force from garment 302 may improve patient comfort when compared to electrodes held in place by adhesives. Though depicted as four sensing electrodes and three therapy electrodes in the example of FIG. 3, garment 302 may include any number of sensing, or therapy electrodes. The number and location of electrodes may depend on patient anatomy as well as a configuration to provide the best signal quality.

The example of FIG. 3 depicts sensing electrodes 342 and 346 as substantially circular disk shaped electrodes and sensing electrodes 344 and 340 as substantially circular ring shaped electrode surrounding electrodes 342 and 346. For example, FIG. 3 depicts sensing electrode 342 as a disk surrounded by the ring of electrode 340. In other examples, electrodes 340, 342, 344 and 346 may be any substantially circular shape, such as an oval, octagon, or similar shape. In other examples, electrodes 340, 342, 344 and 346 may be other geometric shapes such as square or rectangle. In contrast, other examples of multiple sensing electrodes may have arranged the electrodes that are approximately the same size with equal, or approximately equal spacing between the electrodes.

Electrodes 342 and 340 form a concentric arrangement and may appear to be a Laplacian bipolar type electrode. However, electrodes 342 and 340 connect to the circuitry of system 300 in a completely different manner than the high side and low side of a bipolar type electrode. In this disclosure, electrode 342, as well as electrode 346 connects to circuitry that outputs an impedance measurement signal. In other words, the pair of electrodes 342 and 346 are configured to output the impedance measurement signal. In some examples the impedance measurement signal may be a constant current signal. In other examples, the impedance measurement signal may be a high frequency signal, e.g., approximately 8 kHz-16 kHz.

The third and fourth electrodes 344 and 340 may connect to a high side and low side inputs of an amplifier to measure voltage. In contrast, a bipolar Laplacian electrode would connect the inner disk and outer ring electrodes to the high side and low side of an amplifier to measure voltage. In some examples, the impedance measurement signal may be injected to electrode 342 and return from electrode 346, or vice versa.

Sensing circuitry connected to sensing electrodes 340 and 344 may measure the induced voltage in the patient's tissue caused by the impedance measurement signal. The measured induced voltage may provide an indication to processing circuitry 310 of a biological impedance of the patient's tissue. In some examples, the sensing circuitry may detect if an electrode is not connected to the patient's tissue. For example, if the induced voltage is outside a threshold range, the electrodes may be disconnected from the body.

As shown in FIG. 3, electrodes 344 and 340 may be separated and spaced at different locations relative to heart 312 and may sense electrocardiogram (ECG) signals as well as biological impedance signals from patient 308. Sensing electrodes 340, 342, 344 and 346 may also measure bioelectrical signals related to biological impedance, fluid-status monitoring, heart failure, sleep apnea, ischemia detection, lead connectivity detection (also referred to as lead off detection), as well as cardiac arrhythmia such as atrial fibrillation (AF), ventricular tachycardia (VT), ventricular fibrillation (VF) and so on. In some examples, electrodes 340, 342, 344 and 346 may be located at other positions different than shown in FIG. 3, e.g., lateral, or posterior relative to heart 312.

In some examples, garment 302 may also include a motion sensor, such as an accelerometer or similar sensor (not shown in FIG. 2). One or more motion sensors may be included in processing circuitry 310, and/or located elsewhere in garment 302. The motion sensor of this disclosure may be configured to determine one or more of movement or posture of the patient. For example, patient 308 may increase activity level, such as running, jumping and so on, which may cause increased movement between sensing electrodes 340, 342, 344 and 346 and the patient's skin. Also, patient 308 may change posture from an upright to a sitting or supine position. In some examples, external devices 322 and 326 as well as portable computing device 328 may include sensors that indicate movement, temperature, and so on. Processing circuitry 310 may receive the indication of movement and/or posture of patient 308, and in some examples, may dynamically adjust the compression level of garment 302 based on one or more of the indication of the compression level from the mechanical sensor, the received bioelectrical signals or the indication from the motion sensor.

In operation, processing circuitry of any of the components of system 300, as well as any other system of this disclosure, such as systems 100 and 200 described above in relation to FIGS. 1 and 2, may sense and detect one or more precursors to a tachyarrhythmia and trigger a non-shock response. As described above in relation to FIGS. 1 and 2, a non-shock response may include triggering a second detection technique to confirm the presence of a precursor to tachyarrhythmia. In some examples the initial detection technique may consume relatively lower energy, while the triggered subsequent detection technique, or techniques may use relatively higher energy compared to the initial detection technique. For example, based on results of the first detection algorithm, such as an STV detection algorithm described above in relation to FIG. 1, the processing circuitry of system 300 may trigger a second detection algorithm. The second detection algorithm may run on the same device or a different device. The second detection algorithm may consume relatively more resources than the first algorithm and provide relatively higher sensitivity and/or higher specificity than the first algorithm.

In other examples, a non-shock response may include that the detecting device, e.g., IMD 314, may send a message to an application on a smartphone or similar device of the patient to take one or more actions. For example, IMD 314 may send an electronic message that causes portable computing device 328 to alert patient 308 to take some action to avoid or prevent a possible imminent arrhythmia. Depending on the situation of patient 308, some examples of actions may include an increase in oral antiarrhythmics for a specified period, a self-injection of an antiarrhythmic such as adenosine, atropine, lidocaine and so on, an alert to cease stressful activities or exercise, an alert to cease caffeine intake, an alert to put on a wearable defibrillator, such as garment 302, or to take some other similar action.

One such action may include to notify the patient to apply a low level electro-magnetic field based on detecting a precursor to an arrhythmia. In some examples, applying low level a pulsed electromagnetic field (EMF) in the micro-Gauss range of intensity, may increase the threshold at which an arrhythmia may occur or reduce the duration of an episode. In some examples, applying the EMF to specific anatomical locations, such as to the cervical vagal trunk or the chest area near the heart may provide desirable anti-arrhythmic response in a patient. In some examples, garment 302 may include a device to apply the EMF to patient 308.

In other examples, as described above in relation to FIG. 1, a medical device of system 300 with sensing circuitry configured to sense cardiac activity of a patient, such as IMD 314, may detect a precursor to a ventricular tachyarrhythmia, or other arrhythmia. In response to detecting the precursor to the tachyarrhythmia, the device may output a communication to a second device that may trigger the second device to deliver a non-shock therapy to inhibit the onset of the ventricular tachyarrhythmia. As described above in relation to FIGS. 1 and 2, in some examples second device may include a pump and a fluid reservoir, and the non-shock therapy may include to deliver fluid from the reservoir to the patient. In other examples, the non-shock therapy may include to apply electrical stimulation therapy to neurological tissue, and/or tissue of one or more organs of the patient. An organ may include adrenal glands, liver, or some other organ.

FIG. 4 is a conceptual diagram illustrating neurological tissue of a patient according to one or more techniques of this disclosure. A neurostimulator, which may be connected to a lead (not shown in FIG. 4), may apply electrical stimulation therapy to one or more regions of the patient shown in FIG. 4. In other examples, the neurostimulator may apply electrical stimulation therapy to other neurological tissue of the patient not shown in FIG. 4, including brain tissue stimulation, such as deep brain stimulation (DBS), spinal cord stimulation (SCS), tibial nerve stimulation, subcutaneous nerves stimulation, or some other neurological tissue. The neurostimulator may be an implantable neurostimulator (INS), a wearable device or some similar neurostimulator.

In the example of FIG. 4, the neurostimulator may deliver electrical stimulation therapy via one or more implanted leads (not shown in FIG. 4). In some examples according to this disclosure may include transvascular placement of a lead such that the lead passes from within a blood vessel of patient 412 through a wall of the vessel so that stimulation and sensing electrodes on the lead may terminate adjacent a target nerve tissue stimulation site. Although the example of FIG. 4 describes placement in the context of stimulating vagal nerves in the neck of the patient, in other examples, a lead, and any attached electrodes, may also be arranged for vagal nerve stimulation in, e.g., the thorax, and/or adjacent to the esophagus, or other nerves such as the thoracic nerve.

Extravascular lead placement techniques according to this disclosure provide placement of leads for nerve tissue stimulation and/or nerve signal sensing using implantation procedures with reduced invasiveness and without the need to anchor the leads at or near their distal end. In general, the disclosed techniques include placing a portion of a medical lead having an electrode in an extravascular space within a sheath of tissue within patient 412, and adjacent nerve tissue that is also within the sheath of tissue. The lead is anchored offset from the electrode at least partially outside of the sheath.

FIG. 4 illustrates vagus nerve 450 including many branches, such as pharyngeal and laryngeal branches 452, cardiac branches 454, as well as the gastric and pancreaticoduodenal branches (not specifically labeled in FIG. 4). Vagus nerve 450 originates in the brainstem, runs in the neck through carotid sheath 156 with jugular vein 158 and common carotid artery 160, and then adjacent to the esophagus to the thoracic and abdominal viscera.

Vagus nerve 450 provides the primary parasympathetic nerve to the thoracic and most of the abdominal organs. For example, vagus nerve 450 provides parasympathetic innervation to the heart, and stimulation of the nerve has been demonstrated to drive the parasympathetic nervous system and thereby overcome an accelerated sympathetic tone, which may be exhibited by patients suffering from various tachycardia conditions, as well as heart failure. In one such tachycardia application, the efferent fibers of the vagus nerve, such as one or more superior and/or inferior cardiac branches may be electrically stimulated to manage the accelerated arrhythmia. Vagal nerve stimulation may also have afferent effects that result in nerve reflex changes that affect heart rate. In addition to heart innervations, vagus nerve 450 is responsible for such varied tasks as gastrointestinal peristalsis, sweating, as well as muscle movements related to speech. Electrical stimulation of vagus nerve 450 may be useful in treating, not only heart failure and arrhythmia conditions, but also various other conditions including, e.g., depression, epilepsy, and various gastrointestinal conditions.

In other examples, in response to a trigger from a cardiac monitoring device that detects a precursor to an arrhythmia, a neurostimulator may apply electrical stimulation therapy to stellate ganglion 440. For example, short-term variability in some patients may increase with left stellate ganglion activity. An indication of STV may be used by a system of this disclosure to titrate output from an implantable neurostimulator that could deliver a pulse sequence to create a stellate ganglion block. A stellate ganglion block ablation is one intervention for an intractable VT storm.

In other examples, a single device may connect to both cardiac monitoring and therapy delivery electrodes and neurological monitoring and therapy delivery electrodes (not shown in FIG. 4). The single device may connect with one or more cardiac leads that include electrodes for sensing and/or therapy, as well as one or more neural leads with sensing and/or therapy electrodes. The single device may perform any of the functions or combinations of functions described herein, such as using a detection technique that consumes relatively less resources to trigger one or more additional detection techniques that may consume relatively more resources and may deliver relatively higher sensitivity and/or higher specificity to determine whether the patient is indicating a precursor to an arrhythmia.

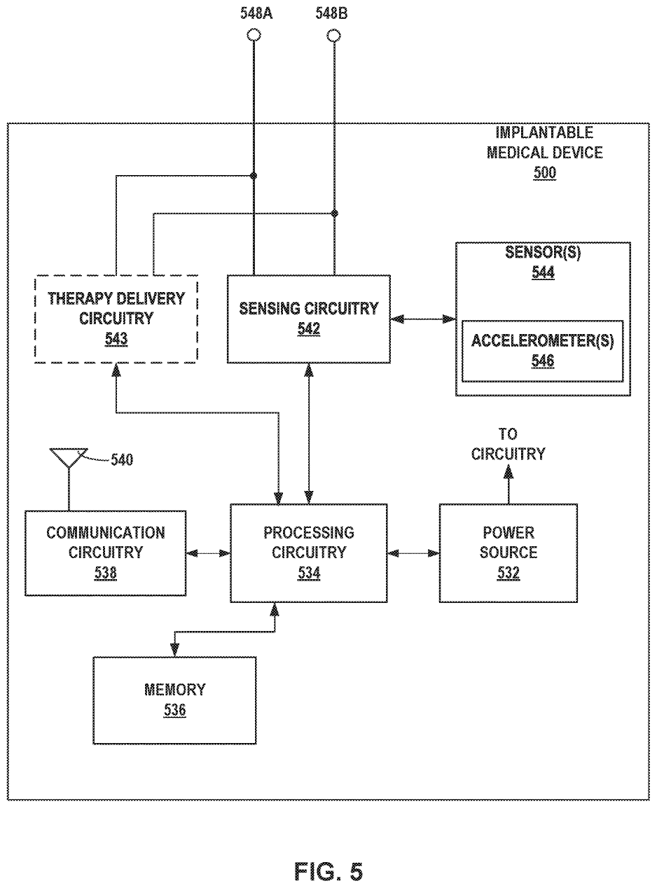

FIG. 5 is a block diagram illustrating an example medical device according to one or more techniques of this disclosure. IMD 500 of FIG. 5 illustrates one possible example configuration of IMD 14 or IMD 12 of medical system 10 of FIG. 1 as well as IMD 209 and IMD 251 of FIG. 2, and IMD 314 and garment 302 of FIG. 3 and devices associated with the operation described in relation to FIG. 4.

In the illustrated example, IMD 500 includes power source 532, processing circuitry 534, memory 536, communication circuitry 538, communication antenna 540, sensing circuitry 542, sensor(s) 544, therapy delivery circuitry 543, and electrodes 548A and 548B (collectively, “electrodes 548”). Although the illustrated example includes two electrodes 548, in other examples IMD 500 may be coupled to more than two electrodes 548.

Processing circuitry 534 may include fixed function circuitry and/or programmable processing circuitry. Processing circuitry 534 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 534 may include multiple components, such as any combination of one or more microprocessors, one or more controllers, one or more DSPs, one or more ASICs, or one or more FPGAs, as well as other discrete or integrated logic circuitry. The functions attributed to processing circuitry 534 herein may be embodied as software, firmware, hardware, or any combination thereof.

Sensing circuitry 542 is coupled to electrodes 548. Sensing circuitry 542 may sense signals from electrodes 548, e.g., to produce a cardiac EGM, to facilitate monitoring the electrical activity of the heart. Processing circuitry 534 may receive indications from sensing circuitry 542 to determine heart rates or heart rate variability, or to detect arrhythmias (e.g., tachyarrhythmias or bradycardia), patient breathing rhythm, biological impedance, or other bioelectrical signals via electrodes 548 and other metrics or conditions described above in relation to FIGS. 1-4. Sensing circuitry 542 also may monitor signals from sensors 544, which may include one or more accelerometers, pressure sensors, temperature sensors and/or optical sensors, as examples. In some examples, sensing circuitry 542 may include one or more filters and amplifiers for filtering and amplifying signals received from electrodes 548 and/or sensors 544.

Sensing circuitry 542 and/or processing circuitry 534 may be configured to detect cardiac depolarizations (e.g., P-waves of atrial depolarizations or R-waves of ventricular depolarizations) when the cardiac EGM amplitude crosses a sensing threshold. For cardiac depolarization detection, sensing circuitry 542 may include a rectifier, filter, amplifier, comparator, and/or analog-to-digital converter, in some examples. In some examples, sensing circuitry 542 may output an indication to processing circuitry 534 in response to sensing of a cardiac depolarization. In this manner, processing circuitry 534 may receive detected cardiac depolarization indicators corresponding to the occurrence of detected R-waves and P-waves in the respective chambers of heart. Processing circuitry 534 may use the indications of detected R-waves and P-waves for determining inter-depolarization intervals, heart rate, and detecting arrhythmias, such as tachyarrhythmias, bradyarrhythmias, and asystole.

Sensing circuitry 542 may also provide one or more digitized cardiac EGM signals to processing circuitry 534 for analysis, e.g., for use in cardiac rhythm discrimination. In some examples, processing circuitry 534 may store the digitized cardiac EGM in memory 536. Processing circuitry 534 of IMD 500, and/or processing circuitry of another device that retrieves data from IMD 500, may analyze the cardiac EGM.

In some examples, IMD 500 may include therapy delivery circuitry 543.

Therapy delivery circuitry 543 may be configured to output electrical stimulation therapy to target tissue of the patient, such as to cardiac tissue, nerve tissue and similar patient tissue. In other examples, therapy delivery circuitry 543 may include a drug pump that provides anti-arrhythmia medication, outputs the EMF therapy or other functions described herein. In some examples, processing circuitry 534 may control one or more parameters of electrical stimulation from therapy delivery circuitry 543 based on bioelectrical signals sensed by sensing circuitry 542. For example, processing circuitry 534 may determine that ventricular contraction is later than expected, e.g., a duration since a previous contraction exceeds a duration threshold. Processing circuitry may cause therapy deliver circuitry to output electrical stimulation therapy in the form of a pacing pulse to cause the heart of the patient to contract, as well as any of the therapy delivery described above in relation to FIGS. 1-4.

Communication circuitry 538 may include any suitable hardware, firmware, software, or any combination thereof for communicating with another device, such as external computing device 22, another networked computing device, or another IMD or sensor. Under the control of processing circuitry 534, communication circuitry 538 may receive downlink telemetry from, as well as send uplink telemetry to external computing device 22 or another device with the aid of an internal or external antenna, e.g., antenna 540. In addition, processing circuitry 534 may communicate with a networked computing device via an external device (e.g., external computing device 22 of FIG. 1) and a computer network, such as the Medtronic CareLink® Network. Antenna 540 and communication circuitry 538 may be configured to transmit and/or receive signals via inductive coupling, electromagnetic coupling, Near Field Communication (NFC), Radio Frequency (RF) communication, Bluetooth, Wi-Fi, or other proprietary or non-proprietary wireless communication schemes. IMD 500 may receive messages from external computing device 22, another medical device worn, or implanted in, patient 26 (of FIG. 1) or from some other source, which may cause IMD 500 to take a measurement via the electrodes, or other sensors, or to deliver electrical stimulation therapy. In some examples, processing circuitry 534 may cause communication circuitry 538 to output a communication to trigger a second device to provide non-shock therapy. Similarly, communication circuitry 538 may receive an indication that triggers processing circuitry 534 to cause therapy delivery circuitry 543 to deliver a non-shock therapy as described above.

In some examples, memory 536 includes computer-readable instructions that, when executed by processing circuitry 534, cause IMD 500 and processing circuitry 534 to perform various functions attributed to IMD 500 and processing circuitry 534 herein.

Memory 536 may include any volatile, non-volatile, magnetic, optical, or electrical media, such as a random-access memory (RAM), read-only memory (ROM), non-volatile RAM (NVRAM), electrically erasable programmable ROM (EEPROM), flash memory, or any other digital media. Memory 536 may store, as examples, programmed values for one or more operational parameters of IMD 500 and/or data collected by IMD 500, e.g., posture, heart rate, activity level, respiration rate, therapy delivery statistics, and other parameters, as well as digitized versions of physiological signals sensed by IMD 500, for transmission to another device using communication circuitry 538.

In the example of FIG. 5, power source 532 that may be coupled to the electronic circuitry provided in IMD 500 and is configured to provide electrical power to these circuits outside of a charging session, e.g., when not receiving wireless power from a primary coil. Power source 532 may, for example, be a primary (non-rechargeable) or rechargeable battery.

In the illustrated example, IMD 500 includes processing circuitry 534 and an associated memory 536, sensing circuitry 542, therapy delivery circuitry 543, one or more sensors 544, and the communication circuitry 538 coupled to antenna 540 as described above. However, IMD 500 need not include all of these components, or may include additional components.

In operation, IMD 500 and any of the systems and components described above in relation to FIGS. 1-5, may perform any of the functions, or combinations of functions, described in this disclosure. For example, IMD 500 may be configured to reduce energy consumption from power source 532 by running at low power when possible and running higher energy functions when needed, e.g., based on detection of a triggering event. For example, processing circuitry 534 may execute any of STV algorithms, or some other algorithm, for a short period of time each day and use that measurement to determine how frequently and/or for how long the algorithm should be on for the next 24 hours. In other words, the triggering event to run a precursor detection scheme may be a predetermined time interval, e.g., a day, a week, a period of hours or minutes or some similar time interval. Alternatively, the triggering event may trigger a first detection algorithm, such that processing circuitry 534, or some other processing circuitry of systems 10, 200 or 300 described above, may execute the first detection algorithm. Then, based on results of the first detection algorithm, the processing circuitry may execute a second detection algorithm that may consume relatively more resources than the first detection algorithm, but may provide relatively higher sensitivity and/or higher specificity than the first detection algorithm. In some examples the second detection algorithm may also consider different factors than the first algorithm to confirm or deny the presence of an arrhythmia precursor. Along with any of the STV algorithms described above, other examples of detection algorithms may include artificial intelligence (AI), and periodic repolarization dynamics (PRD).

In some examples, processing circuitry 534, or processing circuitry of systems 10, 200 and 300, may execute any of several algorithms and other programming instructions to perform the functions described above in relation to FIGS. 1-4. Some examples of initial metrics, or first algorithms used to trigger a second algorithm may include determining whether the PVC burden exceeds a PVC burden threshold, as described above. In other examples, processing circuitry 534 may determine PVC coupling intervals and/or compensatory pauses, e.g., compared to a coupling interval threshold. In some examples either, or both of multi-focal PVC and of mono-focal PVCs may act as a triggered mechanism. In other examples, processing circuitry of the systems of this disclosure may determine whether the number of NSVT per day and/or the number of NSVT beats per day satisfies a threshold or in other examples the NSVT coupling interval is used. In other examples, the processing circuitry may use other intervals such as half a day, multiple days, or some other interval. As described above in relation to FIGS. 1 and 2 any computing device in network 250 may analyze device parameters to trigger STV measurements. In other examples, as described above in relation to FIG. 2, processing circuitry of this disclosure may run an STV algorithm for a short period of time each day, e.g., in the early morning, and use that measurement (and changes with respect to previous days) to determine how frequently and/or for how long the algorithm should be on for the next 24 hours, or for some other interval. In other examples, as described above in relation to FIG. 1, the processing circuitry may use T-wave alternans and/or T-wave morphology changes over a short period of time, e.g., in the early morning, and use this measurement in combination with the prior daily trends to determine how frequently and/or for how long the algorithm should be on for some future interval.

As described above, the processing circuitry of this disclosure may use any combination described above to either trigger some follow-up measurement, or to directly trigger some non-shock therapy described above. For example, the processing circuitry may use a combination of PVC burden and a NSVT metric to trigger either a follow-up measurement such as an STV algorithm to confirm a tachyarrhythmia precursor, or to trigger some non-shock therapy. In other examples, the processing circuitry may use any combination of the above metrics as well as patient-specific information such as comorbidities, age, gender, BMI, right ventricular ejection fraction (RVEF), left ventricular ejection fraction (LVEF), other structural heart disease (SHD) or some other patient information to customize the device response to a possible tachyarrhythmia precursor.

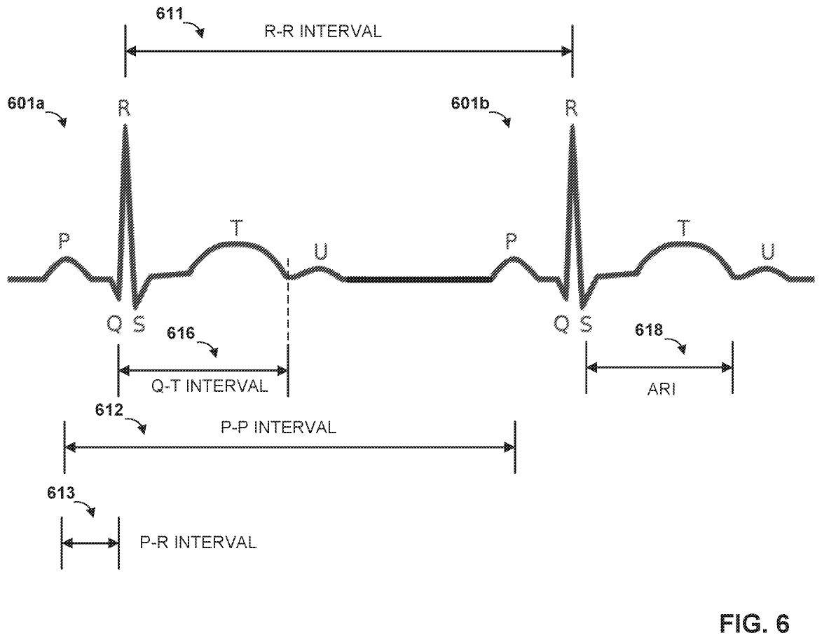

FIG. 6 shows one example of an ECG signal that may be sensed, for example, by an IMD 12 and IMD 14 as described above in relation to FIG. 1. As depicted, each individual cardiac cycle 601a, 601b within ECG signal includes distinguishable characteristics. For example, the cardiac cycles of FIG. 6 include P, Q, R, S, T and U waves or characteristics. One or more of these ECG signal characteristics may be processed and/or analyzed to determine one or more indications of a patient's health, for example to determine at least one interval duration and/or to determine at least one heart rate signal that includes at least one indication of at least one duration interval.

Processing circuitry of the systems of this disclosure may be adapted to detect an occurrence of an R-wave of an ECG signal that represents a cardiac cycle by one or more sense amplifiers as discussed above with respect to FIG. 4. R-waves may be utilized by processing circuitry of this disclosure, e.g., of IMD 500, to determine one or more interval durations that represent a timing of cardiac cycles. In one example, an interval duration may be determined based on an R-R interval 611, or an amount to time between detection of consecutive R-waves as shown in FIG. 6. Other characteristics of an ECG signal may instead be detected for the purpose of determining an interval duration, for example a P-P interval 612 or a P-R interval 613 as also depicted in FIG. 6. Variability in Q-T interval 616 and S-T interval 618 may provide information of a precursor to a possible tachyarrhythmia. S-T interval 618 may also be referred to as activation recovery interval 618 in this disclosure.

In some cases, a patient's heart rate may include interval durations that are longer or shorter than others. As described above, interval durations of a patient's heart rate may be processed and/or analyzed, and variations in intervals durations, also referred to as Heart Rate Variability (HRV), may be utilized by an internal or external medical device, physician, or other user to predict or detect one or more autonomic conditions of a patient. In addition, in response to detection, processing, and/or analysis of HRV of a patient's heart rate, one or more various therapies may be initiated or titrated to remedy or improve one or more detected autonomic conditions, as described above. Also, as described above in relation to FIG. 1, processing circuitry of the medical systems of this disclosure may analyze variability in amplitude, morphology, and other factors along with temporal variability.

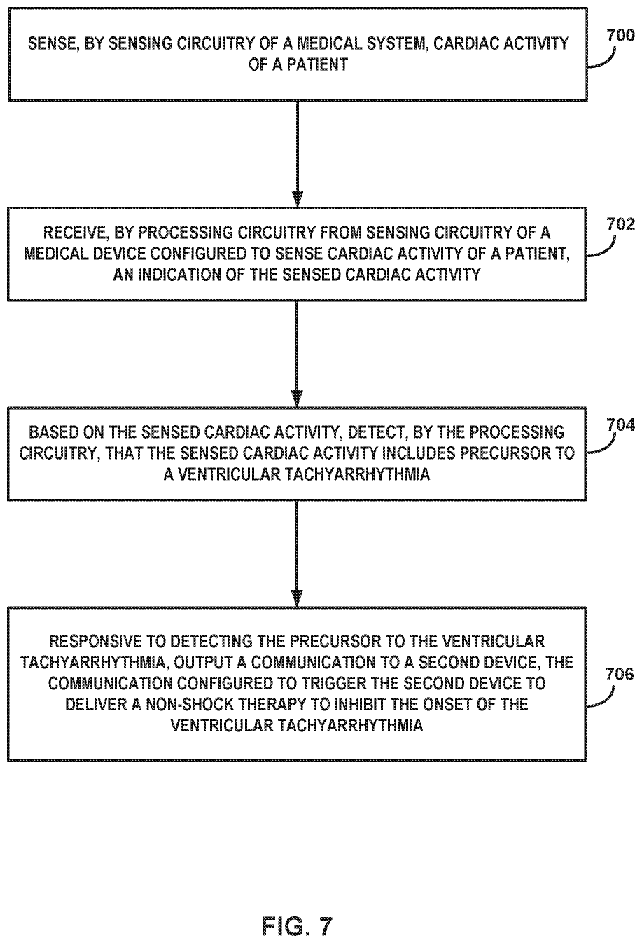

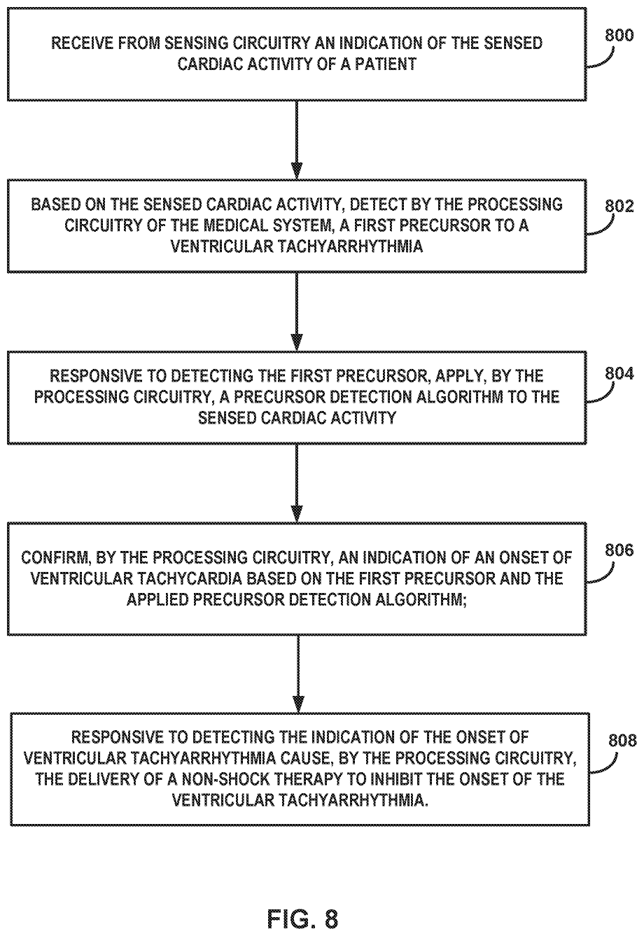

FIG. 7 is a flow chart illustrating an example operation of a system according to one or more techniques of this disclosure. The processing circuitry in the description of FIG. 7 may refer to processing circuitry in one of the components of a system of this disclosure in some examples. In other examples, one or more steps in the blocks of FIG. 7, or portions of a block of FIG. 7, may be distributed among several components, e.g., some sensing may be done by IMD 12 of FIG. 1, while some of the analysis may be done by servers 24.

As shown in FIG. 7, sensing circuitry of a medical system of this disclosure may sense cardiac, and/or neurological activity of a patient (700). The sensing circuitry may receive bioelectrical signals from patient 26 via any of the electrodes, or other sensors on the devices described above in relation to FIGS. 1-5, e.g., electrodes 548A and 548B, accelerometers 549, depicted in FIG. 5, hemodynamic sensors, temperatures sensors and other sensors (not shown in FIG. 7).

Processing circuitry of one or more of the components of a system of this disclosure may receive an indication of the sensed cardiac activity from sensing circuitry of a medical device configured to sense cardiac activity of a patient, e.g., IMD 500 of FIG. 5 (702). Based on the sensed cardiac activity, the processing circuitry may detect a precursor to a ventricular tachyarrhythmia (704). In some examples, detecting the precursor to the ventricular tachyarrhythmia comprises detecting a triggering event, such as short-term variability in ARI 618 of FIG. 7, the expiration of a timer or any of the other precursors described above in relation to FIGS. 1-5.

Responsive to detecting the triggering event, the processing circuitry may apply a precursor detection algorithm to the sensed cardiac activity (704). In some examples, applying the precursor detection algorithm may include an analysis of a recorded and stored EGM of the cardiac activity. In other examples, the processing may also, or instead, apply the precursor detection algorithm to analyze ongoing cardiac activity.

As described, in some examples, a single device of the medical system, e.g., processing circuitry 534 of IMD 500 depicted in FIG. 5, may receive the sensed cardiac activity, detect the precursor, or other triggering event, and apply further analysis to the cardiac activity. In other examples, a processing circuitry of a first device may detect the first precursor and output a communication to a second device to apply a second precursor detection algorithm. In some examples, the “second” precursor detection algorithm may be the same algorithm, e.g., metrics based on NSVT, PVC burden or other examples described above. However, the different location, different sensors, or different timing for the second device may result in a different analysis.

Responsive to detecting the precursor to the ventricular tachyarrhythmia, by the processing circuitry may cause the delivery of a non-shock therapy to inhibit an onset of the ventricular tachyarrhythmia (706). In some examples, the processing circuitry may output a communication to a second device The communication may be configured to trigger the second device to deliver a non-shock therapy to inhibit the onset of the ventricular tachyarrhythmia, such as overdrive pacing, fluid delivery, a low-level electrical field, a patient alert, or some other activity.

FIG. 8 is a flow chart illustrating an example operation of a system configured to perform a multi-level precursor analysis according to one or more techniques of this disclosure. As described above in relation to FIG. 7, The processing circuitry in the description of FIG. 8 may refer to processing circuitry in one or more of the components of a system of this disclosure in some examples. In other examples, one or more steps in the blocks of FIG. 8, or portions of a block of FIG. 8, may be distributed among several components. Also, as with the steps of FIG. 7, the description of FIG. 8 may focus on the example of FIG. 5 to simplify the explanation, however, any of the medical systems configured to sense cardiac activity of a patient, and described above in relation to FIGS. 1-5 may perform the steps of FIGS. 7 and 8.

In the example of FIG. 8, processing circuitry 534 may receive from sensing circuitry 542 an indication of the sensed cardiac activity, e.g., of heart 17 for patient 26 depicted in FIG. 1 (800). Based on the sensed cardiac activity, by the processing circuitry of the medical system may detect a first precursor to a ventricular tachyarrhythmia (802). As described above, the precursor may include a variety of indications based on analysis of the sensed cardiac signal. In some examples, the analysis may take place over a longer time, e.g., looking for patterns over several hours, days, weeks, or some other interval. In other examples, the analysis may be based on a more acute time period, such as a few minutes or seconds. The analysis may include comparing a PVC burden to a PVC burden threshold, or some similar analysis of PVC tracking. Other examples precursors may include variability in the cardiac cycle, an analysis of NSVT metrics, as well as neurological signals described above in relation to FIG. 4.

Responsive to detecting the first precursor, the processing circuitry may apply a precursor detection algorithm to the sensed cardiac activity (804). In some examples, the precursor detection algorithm may include the same or similar analysis as for detecting the first precursor. In other examples, the applied precursor detection algorithm may be a different algorithm. In some examples, consumes relatively more resources than the first precursor detection algorithm, and may provide relatively higher sensitivity and/or higher specificity than the first precursor detection algorithm. In some examples, the processing circuitry may simply perform the same precursor analysis more frequently, and/or for a longer duration, which may consume more resources, e.g., microprocessor wake-up time. For example, the processing circuitry may apply the precursor detection algorithm to the sensed cardiac activity based on a predetermined time interval. Based on results of the first detection algorithm, the processing circuitry execute the first detection algorithm more frequently than the predetermined time interval. In other examples, the processing circuitry may apply a second precursor detection algorithm, or set of algorithms, wherein the second precursor detection algorithms consumes relatively more resources than the first precursor detection algorithm. In this manner the system may conserve battery power while accurately and reliably detecting and acting on precursors to a tachy arrhythmia.

Based on the first precursor and the applied precursor detection algorithm, the processing circuitry may confirm an indication of an imminent onset of ventricular tachycardia (806). As described above, the R-R interval, and other measures of the cardiac cycle of the patient may not yet indicate a heart rhythm in the tachycardia range, but may indicate that an arrhythmia is developing or imminent.

Responsive to detecting the indication of the imminent onset of ventricular tachyarrhythmia cause, by the processing circuitry, the delivery of a non-shock therapy to inhibit the onset of the ventricular tachyarrhythmia (808). As described above in relation to FIGS. 1-4, in some examples, all the steps of FIG. 8 may be performed by a single device. A single device may, detect the precursor, confirm the precursor, and deliver any one of the non-shock therapies described above, e.g., over drive pacing, neurological stimulation, and/or fluid delivery. In other examples, two or more devices of the medical system may perform any one or more steps listed in FIG. 8.

The techniques of this disclosure may also be described in the following examples.

Example 1. A system comprising: sensing circuitry configured to sense cardiac activity of a patient; and processing circuitry configured to: based on the sensed cardiac activity, detect a precursor to a ventricular tachyarrhythmia; and responsive to detecting the precursor to the ventricular tachyarrhythmia, deliver a non-shock therapy to inhibit an onset of the ventricular tachyarrhythmia.

Example 2. The system of example 1, wherein the non-shock therapy comprises any one of: overdrive pacing or high rate pacing.

Example 3. The system of examples 1 and 2, wherein the non-shock therapy comprises refractory period stimulation to the patient.

Example 4. The system of any of examples 1-3, wherein the non-shock therapy comprises to apply electrical stimulation therapy to neurological tissue of the patient.

Example 5. The system of any of any of examples 1-4, wherein the non-shock therapy is anti-arrhythmia neurological stimulation, and wherein the neurological tissue of the patient comprises one or more of: vagal nerve, stellate ganglion, spinal cord stimulation and a thoracic nerve.

Example 6. The system of any of examples 1-5, wherein the non-shock therapy comprises to apply a low-level electrical field to the patient.

Example 7. The system of example 1-6, wherein the non-shock therapy comprises to deliver fluid from a reservoir of a pump to the patient.

Example 8. The system of any of any of examples 1-7 comprising an implantable medical device (IMD), wherein the IMD comprises at least a portion of the sensing circuitry and the processing circuitry, and wherein the IMD is configured to: detect the precursor to the ventricular tachyarrhythmia; and deliver the non-shock therapy.

Example 9. The system of example 8, wherein the IMD comprises a cardiovascular implantable electronic device (CIED) configured to deliver the one or more of overdrive pacing or high rate pacing to the patient.

Example 10. The system of any of any of examples 1-7, comprising: a first medical device configured to apply the non-shock therapy to inhibit the onset of ventricular tachyarrhythmia; a second device comprising at least a portion of: the sensing circuitry configured to sense cardiac activity of a patient; communication circuitry of the system, wherein the communication circuitry is configured to communicate between the first device and the second device; and the processing circuitry, wherein the processing circuitry of the second device is configured to: based on the sensed cardiac activity, detect a precursor to a ventricular tachyarrhythmia; and responsive to detecting the precursor to the ventricular tachyarrhythmia, output a communication to the first device, the communication configured to trigger the first device to apply the non-shock therapy to inhibit the onset of the ventricular tachyarrhythmia.

Example 11. The system of example 10, wherein the second medical device is an implantable medical device (IMD).

Example 12. The system of example 11, wherein the first medical device comprises a cardiovascular implantable electronic device (CIED) configured to provide the one or more of overdrive pacing or high rate pacing to the patient.

Example 13. The system of example 10, wherein the first medical device is a wearable medical device.

Example 14. The system of example 10, wherein the first medical device is an external computing device, wherein the non-shock therapy from the external computing device is configured to alert the patient via a user interface, and wherein the alert comprises one or more of: a recommendation to increase in oral antiarrhythmics for a predetermined duration, a notice for an immediate self-injection of an antiarrhythmic substance including: adenosine, atropine, or lidocaine, a recommendation to cease stressful activities or exercise, a recommendation to cease caffeine intake, or a notice to put on a wearable cardiac defibrillator.

Example 15. The system of any of any of examples 1-14, wherein the precursor to ventricular tachyarrhythmia comprises a short-term variability (STV) in an activation recovery interval (ARI).

Example 16. The system of any of any of examples 1-15, wherein the precursor to ventricular tachyarrhythmia comprises a variability in any portion of the sensed cardiac activity for any one or more of: variability in amplitude in any portion of the sensed cardiac activity or variability in morphology of the sensed cardiac activity.

Example 17. The system of any of any of examples 1-16, wherein the processing circuitry is configured to determine a pre-ventricular contraction (PVC) burden of the patient based on the sensed cardiac activity, and wherein the precursor comprises a PVC burden that exceeds a PVC burden threshold.

Example 18. The system of any of any of examples 1-17, wherein to detect a precursor to a ventricular tachyarrhythmia comprises applying a precursor detection algorithm to the sensed cardiac activity; and wherein the processing circuitry is configured to apply the precursor detection algorithm in response to detecting a triggering event.

Example 19. The system of any of any of examples 1-18, wherein the processing circuitry is configured to determine a pre-ventricular contraction (PVC) burden of the patient based on the sensed cardiac activity, and wherein the triggering event comprises the PVC burden exceeding a PVC burden threshold.

Example 20. The system of any of example 1-19, wherein the triggering event comprises a metric related to non-sustained ventricular tachyarrhythmia (NSVT) events.

Example 21. The system of any of any of examples 1-20, wherein the precursor detection algorithm comprises a first detection algorithm, wherein the triggering event comprises a predetermined time interval and the processing circuitry executes the first detection algorithm based on the predetermined time interval; and wherein, based on results of the first detection algorithm, the processing circuitry executes the first detection algorithm more frequently than the predetermined time interval.

Example 22. The system of any of any of examples 1-21, wherein the precursor detection algorithm comprises a first detection algorithm, wherein, based on results of the first detection algorithm, the processing circuitry executes a second detection algorithm, and wherein the second detection algorithm: consumes relatively more resources than the first detection algorithm, and delivers relatively higher sensitivity and/or higher specificity than the first detection algorithm.

Example 23. The system of any of any of examples 1-22, wherein the second detection algorithm is a short-term variability (STV) algorithm.

Example 24. The system of any of any of examples 1-23, wherein the sensing circuitry comprises a hemodynamic monitor.

Example 25. A method comprising: sensing, by sensing circuitry of a medical system, cardiac activity of a patient; receiving, by processing circuitry of the medical system, an indication of the cardiac activity of the patient; based on the sensed cardiac activity, detecting, by the processing circuitry, that the sensed cardiac activity includes precursor to a ventricular tachyarrhythmia; and responsive to detecting the precursor to the ventricular tachyarrhythmia, causing, by the processing circuitry, the delivery of a non-shock therapy to inhibit an onset of the ventricular tachyarrhythmia.

Example 26. The method of example 25, wherein the non-shock therapy comprises any one of overdrive pacing or high rate pacing.

Example 27. The method of any of examples 25 and 26, wherein the non-shock therapy comprises refractory period stimulation to the patient.

Example 28. The method of any of claims 25-27, wherein the non-shock therapy comprises applying electrical stimulation therapy to neurological tissue of the patient.

Example 29. The method of example 25-28, wherein the non-shock therapy is anti-arrhythmia neurological stimulation, and wherein the neurological tissue of the patient comprises one or more of: vagal nerve, stellate ganglion, spinal cord stimulation and a thoracic nerve.

Example 30. The method of any of examples 25-29, wherein the non-shock therapy comprises applying a low-level electrical field to the patient.

Example 31. The method of example 25-30, wherein the non-shock therapy comprises delivering fluid from a reservoir of a pump to the patient.

Example 32. The method of any of any of examples 25-31 comprising an implantable medical device (IMD), wherein the IMD comprises at least a portion of the sensing circuitry and the processing circuitry, and the method further comprising: detecting, by the IMD, the precursor to the ventricular tachyarrhythmia; and delivering, by the IMD, the non-shock therapy.

Example 33. The method of example 32, wherein the IMD comprises a cardiovascular implantable electronic device (CIED), the method further comprising delivering, by the IMD, the one or more of overdrive pacing or high rate pacing to the patient.

Example 34. The method of any of any of examples 25-33, comprising: a first medical device configured to apply the non-shock therapy to inhibit the onset of ventricular tachyarrhythmia; a second device comprising at least a portion of: the sensing circuitry configured to sense cardiac activity of a patient; communication circuitry of the medical system, wherein the communication circuitry is configured to communicate between the first device and the second device; and the processing circuitry, the method further comprising: based on the sensed cardiac activity, detecting, by processing circuitry of the second device, a precursor to a ventricular tachyarrhythmia; and responsive to detecting the precursor to the ventricular tachyarrhythmia, causing, by processing circuitry of the second device, a communication to the first device, the communication configured to trigger the first device to apply the non-shock therapy to inhibit the onset of the ventricular tachyarrhythmia.

Example 35. The method of example 34, wherein the second medical device is an implantable medical device (IMD).

Example 36. The method of example 35, wherein the first medical device comprises a cardiovascular implantable electronic device (CIED), the method further comprising, delivering, by the first medical device, the one or more of overdrive pacing or high rate pacing to the patient.

Example 37. The method of example 34, wherein the first medical device is a wearable medical device.

Example 38. The method of example 34, wherein the first medical device is an external computing device, wherein the non-shock therapy from the external computing device is configured to alert the patient via a user interface, and wherein the alert comprises one or more of: a recommendation to increase in oral antiarrhythmics for a predetermined duration, a notice for an immediate self-injection of an antiarrhythmic substance including: adenosine, atropine, or lidocaine, a recommendation to cease stressful activities or exercise, a recommendation to cease caffeine intake, or a notice to put on a wearable cardiac defibrillator.

Example 39. The method of any of any of examples 25-38, wherein the precursor to ventricular tachyarrhythmia comprises a short-term variability (STV) in an activation recovery interval (ARI).

Example 40. The method of any of any of examples 25-39, wherein the precursor to ventricular tachyarrhythmia comprises a variability in any portion of the sensed cardiac activity for any one or more of: variability in amplitude in any portion of the sensed cardiac activity or variability in morphology of the sensed cardiac activity.

Example 41. The method of any of any of examples 25-40, wherein the processing circuitry is configured to determine a pre-ventricular contraction (PVC) burden of the patient based on the sensed cardiac activity, and wherein the precursor comprises a PVC burden that exceeds a PVC burden threshold.

Example 42. The method of any of any of examples 25-41, wherein to detecting a precursor to a ventricular tachyarrhythmia comprises applying a precursor detection algorithm to the sensed cardiac activity, the method further comprising applying, by the processing circuitry of the medical system, the precursor detection algorithm in response to detecting a triggering event.

Example 43. The method of any of any of examples 25-42, the method further comprising determining, by the processing circuitry, a pre-ventricular contraction (PVC) burden of the patient based on the sensed cardiac activity, and wherein the triggering event comprises the PVC burden exceeding a PVC burden threshold.

Example 44. The method of any of example 25-43, wherein the triggering event comprises a metric related to non-sustained ventricular tachyarrhythmia (NSVT) events.

Example 45. The method of any of any of examples 25-44, wherein the precursor detection algorithm comprises a first detection algorithm, wherein the triggering event comprises a predetermined time interval, the method further comprising: executing, by the processing circuitry, the first detection algorithm based on the predetermined time interval; and based on results of the first detection algorithm, executing, by the processing circuitry, the first detection algorithm more frequently than the predetermined time interval.

Example 46. The method of any of any of examples 25-45, wherein the precursor detection algorithm comprises a first detection algorithm, the method further comprising, based on results of the first detection algorithm, executing, by the processing circuitry, a second detection algorithm, and wherein the second detection algorithm: consumes relatively more resources than the first detection algorithm, and delivers relatively higher sensitivity and/or higher specificity than the first detection algorithm.

Example 47. The method of any of any of examples 25-46, wherein the second detection algorithm is a short-term variability (STV) algorithm.

Example 48. The method of any of any of examples 25-47, wherein the sensing circuitry comprises a hemodynamic monitor.

Example 49. A non-transitory computer-readable storage medium comprising instructions that, when executed, cause processing circuitry of a computing device to: receive an indication of cardiac activity of a patient from sensing circuitry of a medical system, wherein: the sensing circuitry is configured to sense cardiac activity of the patient, and the computing device is a component of the medical system; based on the sensed cardiac activity, detect a precursor to a ventricular tachyarrhythmia; and responsive to detecting the precursor to the ventricular tachyarrhythmia, deliver a non-shock therapy to inhibit an onset of the ventricular tachyarrhythmia.

Example 50. A method comprising: receiving, by processing circuitry and from sensing circuitry of a medical system configured to sense cardiac activity of a patient, an indication of the sensed cardiac activity; based on the sensed cardiac activity, detecting by the processing circuitry of the medical system, a first precursor to a ventricular tachyarrhythmia, responsive to detecting the first precursor, applying, by the processing circuitry, a precursor detection algorithm to the sensed cardiac activity; and confirming, by the processing circuitry, an indication of an onset of ventricular tachycardia based on the first precursor and the applied precursor detection algorithm; responsive to detecting the indication of the onset of ventricular tachyarrhythmia causing, by the processing circuitry, the delivery of a non-shock therapy to inhibit the onset of the ventricular tachyarrhythmia.

Example 51. The method of example 50, wherein the medical system comprises an implantable medical device (IMD), wherein the IMD comprises at least a portion of the sensing circuitry and the processing circuitry, and the method further comprising: detecting, by the IMD, the first precursor to the ventricular tachyarrhythmia; applying, by the IMD, the precursor detection algorithm, and delivering, by the IMD, the non-shock therapy.

Example 52. The method of examples 50 and 51, wherein the IMD comprises a cardiovascular implantable electronic device (CIED), the method further comprising, delivering, by the CIED, the non-shock therapy comprising one or more of refractory period stimulation, overdrive pacing or high rate pacing to the patient.

Example 53. The method of any of any of examples 50-52 wherein the medical system comprises a first medical device configured to apply the non-shock therapy to inhibit the onset of ventricular tachyarrhythmia; wherein the medical system comprises a second medical device comprising at least a portion of: the sensing circuitry configured to sense cardiac activity of a patient; communication circuitry for the medical system, wherein the communication circuitry is configured to communicate between at least the first device and the second device; and the processing circuitry, wherein the method further comprises: responsive to detecting the first precursor, by the processing circuitry of the second device applying, by the processing circuit of the second device, the precursor detection algorithm.

Example 54. The method of any of any of examples 50-53, wherein applying the precursor detection algorithm comprises causing the communication circuitry to output a communication to the first device, receiving, by the first device, the output communication; responsive to receiving the output communication, confirming by the first device, the onset of the ventricular tachyarrhythmia by applying the precursor detection algorithm.

Example 55. The method of any of any of examples 50-54, wherein applying the precursor detection algorithm comprises applying, by the processing circuit of the second device, the precursor detection algorithm, thereby confirming the onset of the ventricular tachyarrhythmia by the second device, the method further comprising causing, by the second device, the communication circuitry to output a communication to the first device, the communication configured to trigger the first device to deliver the non-shock therapy.

Example 56. The method of any of any of examples 50-55, wherein the second medical device is an implantable medical device (IMD).

Example 57. The method of any of any of examples 50-56, wherein the first medical device comprises a cardiovascular implantable electronic device (CIED) configured to provide one or more of refractory period stimulation, overdrive pacing or high rate pacing to the patient.

Example 58. The method of any of any of examples 50-57, wherein the first medical device is a wearable medical device.

Example 59. The method of any of claims 50-58, wherein the non-shock therapy comprises to apply electrical stimulation therapy to neurological tissue of the patient.

Example 60. The method of any of claims 50-59, wherein the non-shock therapy is anti-arrhythmia neurological stimulation, and wherein the neurological tissue of the patient comprises one or more of: vagal nerve, stellate ganglion, spinal cord stimulation and a thoracic nerve.

Example 61. The method of any of claims 50-60, wherein the non-shock therapy comprises to apply a low-level electrical field to the patient.

Example 62. The method of any of claims 50-61, wherein the non-shock therapy comprises to deliver fluid from a reservoir of a pump to the patient.

Example 63. The method of any of claims 50-62, wherein the first medical device is an external computing device, wherein the non-shock therapy from the external computing device is configured to alert the patient via a user interface, and wherein the alert comprises one or more of: a recommendation to increase in oral antiarrhythmics for a predetermined duration, a notice for an immediate self-injection of an antiarrhythmic substance including: adenosine, atropine, or lidocaine, a recommendation to cease stressful activities or exercise, a recommendation to cease caffeine intake, or a notice to put on a wearable cardiac defibrillator.

Example 64. The method of any of any of examples 50-63, wherein detecting the first precursor comprises applying, by the processing circuitry, a second precursor detection algorithm to the sensed cardiac activity; and wherein the precursor detection algorithm comprises a second precursor detection algorithm, wherein the second precursor detection algorithm: consumes relatively more resources than the first precursor detection algorithm, and provides relatively higher sensitivity and/or higher specificity than the first precursor detection algorithm.

Example 65. The method of any of any of examples 50-64, wherein detecting the first precursor comprises applying, by the processing circuitry, a first precursor detection algorithm to the sensed cardiac activity based on a predetermined time interval; and the method further comprising, responsive to the results of the first detection algorithm, executing, by the processing circuitry the first detection algorithm more frequently than the predetermined time interval.

Example 66. The method of any of any of examples 50-65, wherein the first precursor comprises a pre-ventricular contraction (PVC) burden based on the sensed cardiac activity, the method further comprising applying the precursor detection algorithm when the PVC burden exceeds a PVC burden threshold.

Example 67. The method of any of any of examples 50-66, wherein the first precursor comprises a metric related to non-sustained ventricular tachyarrhythmia (NSVT) events.

Example 68. The method of any of any of examples 50-67, wherein the first precursor to the ventricular tachyarrhythmia comprises a short-term variability (STV) in an activation recovery interval (ARI) of the sensed cardiac activity.

Example 69. The method of any of any of examples 50-68, wherein the first precursor to the ventricular tachyarrhythmia comprises a short-term variability (STV) in a QT interval of the sensed cardiac activity.

Example 70. The method of any of any of examples 50-69, wherein the sensing circuitry comprises a hemodynamic monitor.

Example 71. A medical system, the system comprising: sensing circuitry configured to sense cardiac activity of a patient; processing circuitry configured to: receive from the sensing circuitry an indication of the sensed cardiac activity; based on the sensed cardiac activity, detect a first precursor to a ventricular tachyarrhythmia; responsive to detecting the first precursor apply a precursor detection algorithm to the sensed cardiac activity; and confirming, by the processing circuitry, an indication of an onset of ventricular tachycardia based on the first precursor and the applied precursor detection algorithm; responsive to detecting the indication of the onset of ventricular tachyarrhythmia causing the delivery of a non-shock therapy to inhibit the onset of the ventricular tachyarrhythmia.

Example 72. The system of example 71, wherein the system comprises an implantable medical device (IMD), wherein the IMD comprises at least a portion of the sensing circuitry and the processing circuitry of the system, and wherein the IMD is configured to: detect the first precursor to the ventricular tachyarrhythmia; apply the precursor detection algorithm, and deliver the non-shock therapy.

Example 73. The system of any of examples 71-72, wherein the IMD comprises a cardiovascular implantable electronic device (CIED) configured to provide one or more of refractory period stimulation, overdrive pacing or high rate pacing to the patient.

Example 74. The system any of any of examples 71-73, wherein the system comprises a first medical device configured to apply the non-shock therapy to inhibit the onset of ventricular tachyarrhythmia; wherein the system comprises a second medical device comprising at least a portion of: the sensing circuitry configured to sense cardiac activity of a patient; communication circuitry for the system, wherein the communication circuitry is configured to communicate between at least the first device and the second device; and the processing circuitry, wherein the processing circuitry of the second device is configured to: responsive to detecting the first precursor applying the precursor detection algorithm; and responsive to confirming the indication of the onset of ventricular tachycardia, cause the communication circuitry to output a message to the first device to apply the non-shock therapy.

Example 75. The system of any of any of examples 71-74, wherein applying the precursor detection algorithm comprises the processing circuitry of the second device to apply the precursor detection algorithm.

Example 76. The system of any of any of examples 71-75, wherein to apply the precursor detection algorithm comprises to cause the communication circuitry to output a communication to the first medical device, wherein the communication is configured to trigger the first device to apply the precursor detection algorithm, confirming the onset of the ventricular tachyarrhythmia.

Example 77. The system of any of any of examples 71-76, wherein the second medical device is an implantable medical device (IMD).

Example 78. The system of any of any of examples 71-77, wherein the first medical device comprises a cardiovascular implantable electronic device (CIED) configured to provide one or more of refractory period stimulation, overdrive pacing or high rate pacing to the patient.

Example 79. The system of any of any of examples 71-78, wherein the first medical device is a wearable medical device.

Example 80. The system of any of claims 71-79, wherein the non-shock therapy comprises to apply electrical stimulation therapy to neurological tissue of the patient.

Example 81. The system of any of claims 71-80, wherein the non-shock therapy is anti-arrhythmia neurological stimulation, and wherein the neurological tissue of the patient comprises one or more of: vagal nerve, stellate ganglion, spinal cord stimulation and a thoracic nerve.

Example 82. The system of any of claims 71-81, wherein the non-shock therapy comprises to apply a low-level electrical field to the patient.

Example 83. The system of any of claims 71-82, wherein the non-shock therapy comprises to deliver fluid from a reservoir of a pump to the patient.

Example 84. The system of any of claims 71-83, wherein the first medical device is an external computing device, wherein the non-shock therapy from the external computing device is configured to alert the patient via a user interface, and wherein the alert comprises one or more of: a recommendation to increase in oral antiarrhythmics for a predetermined duration, a notice for an immediate self-injection of an antiarrhythmic substance including: adenosine, atropine, or lidocaine, a recommendation to cease stressful activities or exercise, a recommendation to cease caffeine intake, or a notice to put on a wearable cardiac defibrillator.

Example 85. The system of any of any of examples 71-84, wherein detecting the first precursor comprises applying, by the processing circuitry, a first precursor detection algorithm to the sensed cardiac activity; and wherein the precursor detection algorithm comprises a second precursor detection algorithm, wherein the second precursor detection algorithm: consumes relatively more resources than the first precursor detection algorithm, and provides relatively higher sensitivity and/or higher specificity than the first precursor detection algorithm.

Example 86. The system of any of any of examples 71-85, wherein detecting the first precursor comprises applying, by the processing circuitry, a first precursor detection algorithm to the sensed cardiac activity based on a predetermined time interval; and wherein, based on results of the first detection algorithm, executing, by the processing circuitry the first detection algorithm more frequently than the predetermined time interval.

Example 87. The system of any of any of examples 71-86, wherein the first precursor comprises a pre-ventricular contraction (PVC) burden based on the sensed cardiac activity, the system further comprising applying the precursor detection algorithm when the PVC burden exceeds a PVC burden threshold.

Example 88. The system of any of any of examples 71-87, wherein the first precursor comprises a metric related to non-sustained ventricular tachyarrhythmia (NSVT) events.

Example 89. The system of any of any of examples 71-88, wherein the first precursor to the ventricular tachyarrhythmia comprises a short-term variability (STV) in an activation recovery interval (ARI) of the sensed cardiac activity.

Example 90. The system of any of any of examples 71-89, wherein the first precursor to the ventricular tachyarrhythmia comprises a short-term variability (STV) in a QT interval of the sensed cardiac activity.

Example 91. The system of any of any of examples 71-90, wherein the sensing circuitry comprises a hemodynamic monitor.

Example 92. A non-transitory computer-readable storage medium comprising instructions that, when executed, cause processing circuitry of a computing device to: receive an indication of cardiac activity of a patient from sensing circuitry of a medical system, wherein: the sensing circuitry is configured to sense cardiac activity of the patient, and the computing device is a component of the medical system; based on the sensed cardiac activity, detect a first precursor to a ventricular tachyarrhythmia; responsive to detecting the first precursor apply a precursor detection algorithm to the sensed cardiac activity; and confirm an indication of an onset of ventricular tachycardia based on the first precursor and the applied precursor detection algorithm; responsive to detecting the indication of the onset of ventricular tachyarrhythmia cause the delivery of a non-shock therapy to inhibit the onset of the ventricular tachyarrhythmia.

In one or more examples, the functions described above may be implemented in hardware, software, firmware, or any combination thereof. For example, the various components of FIGS. 1 and 2, such as IMD 12, IMD 14, external computing device 22, IMD 209, among others may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

The term “non-transitory” may indicate that the storage medium is not embodied in a carrier wave or a propagated signal. In certain examples, a non-transitory storage medium may store data that can, over time, change (e.g., in RAM or cache). By way of example, and not limitation, such computer-readable storage media, may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Combinations of the above should also be included within the scope of computer-readable media.

Instructions may be executed by one or more processors, such as one or more DSPs, general purpose microprocessors, ASICs, FPGAs, or other equivalent integrated or discrete logic circuitry. Accordingly, the term “processor” and “processing circuitry,” as used herein, such as processing circuitry 534 of FIG. 5, may refer to any of the foregoing structure or any other structure suitable for implementation of the techniques described herein. Also, the techniques could be fully implemented in one or more circuits or logic elements.

The techniques of this disclosure may be implemented in a wide variety of devices or apparatuses, including, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

1. A system comprising:
sensing circuitry configured to sense cardiac activity of a patient; and
processing circuitry configured to:
based on the sensed cardiac activity, detect a precursor to a ventricular tachyarrhythmia; and
responsive to detecting the precursor to the ventricular tachyarrhythmia, deliver a non-shock therapy to inhibit an onset of the ventricular tachyarrhythmia.

2. The system of claim 1, wherein the non-shock therapy comprises one or more of: overdrive pacing, high rate pacing, refractory period stimulation, or electrical stimulation therapy to neurological tissue of the patient.

3. The system of claim 2, wherein the non-shock therapy is anti-arrhythmia neurological stimulation, and wherein the neurological tissue of the patient comprises one or more of: vagal nerve, stellate ganglion, spinal cord stimulation and a thoracic nerve.

4. The system of claim 1, wherein the non-shock therapy comprises to apply a low-level electrical field to the patient.

5. The system of claim 1, comprising:
a first medical device configured to apply the non-shock therapy to inhibit the onset of ventricular tachyarrhythmia;
a second device comprising at least a portion of:
the sensing circuitry configured to sense cardiac activity of a patient;
communication circuitry of the system, wherein the communication circuitry is configured to communicate between the first device and the second device; and
the processing circuitry, wherein the processing circuitry of the second device is configured to:
based on the sensed cardiac activity, detect a precursor to a ventricular tachyarrhythmia; and
responsive to detecting the precursor to the ventricular tachyarrhythmia, output a communication to the first device, the communication configured to trigger the first device to apply the non-shock therapy to inhibit the onset of the ventricular tachyarrhythmia.

6. The system of claim 5, wherein the second medical device is an implantable medical device (IMD), and
wherein the first medical device comprises a cardiovascular implantable electronic device (CIED) configured to provide the one or more of overdrive pacing or high rate pacing to the patient.

7. The system of claim 5,
wherein the first medical device is an external computing device,
wherein the non-shock therapy from the external computing device is configured to alert the patient via a user interface, and
wherein the alert comprises one or more of:
a recommendation to increase in oral antiarrhythmics for a predetermined duration,
a notice for an immediate self-injection of an antiarrhythmic substance including: adenosine, atropine, or lidocaine,
a recommendation to cease stressful activities or exercise,
a recommendation to cease caffeine intake, or
a notice to put on a wearable cardiac defibrillator.

8. The system of claim 1, wherein the precursor to ventricular tachyarrhythmia comprises any one of:
a short-term variability (STV) in an activation recovery interval (ARI), or
a variability in any portion of the sensed cardiac activity for any one or more of: variability in amplitude in any portion of the sensed cardiac activity or variability in morphology of the sensed cardiac activity.

9. The system of claim 1,
wherein the processing circuitry is configured to determine a pre-ventricular contraction (PVC) burden of the patient based on the sensed cardiac activity, and
wherein the precursor comprises a PVC burden that exceeds a PVC burden threshold.

10. The system of claim 1,
wherein to detect a precursor to a ventricular tachyarrhythmia comprises applying a precursor detection algorithm to the sensed cardiac activity; and
wherein the processing circuitry is configured to apply the precursor detection algorithm in response to detecting a triggering event.

11. The system of claim 1,
wherein the processing circuitry is configured to determine a pre-ventricular contraction (PVC) burden of the patient based on the sensed cardiac activity, and
wherein the triggering event comprises the PVC burden exceeding a PVC burden threshold.

12. The system of claim 1, wherein the triggering event comprises a metric related to non-sustained ventricular tachyarrhythmia (NSVT) events.

13. The system of claim 1,
wherein the precursor detection algorithm comprises a first detection algorithm,
wherein the triggering event comprises a predetermined time interval and the processing circuitry executes the first detection algorithm based on the predetermined time interval; and
wherein, based on results of the first detection algorithm, the processing circuitry executes the first detection algorithm more frequently than the predetermined time interval.

14. The system of claim 1,
wherein the precursor detection algorithm comprises a first detection algorithm,
wherein, based on results of the first detection algorithm, the processing circuitry executes a second detection algorithm, and
wherein the second detection algorithm:
consumes relatively more resources than the first detection algorithm, and
delivers relatively higher sensitivity and/or higher specificity than the first detection algorithm.

15. The system of claim 1, wherein the second detection algorithm is a short-term variability (STV) algorithm.

16. A method comprising:
sensing, by sensing circuitry of a medical system, cardiac activity of a patient;
receiving, by processing circuitry of the medical system, an indication of the cardiac activity of the patient;
based on the sensed cardiac activity, detecting, by the processing circuitry, that the sensed cardiac activity includes precursor to a ventricular tachyarrhythmia; and
responsive to detecting the precursor to the ventricular tachyarrhythmia, causing, by the processing circuitry, delivery of a non-shock therapy to inhibit an onset of the ventricular tachyarrhythmia.

17. The method of claim 16 comprising an implantable medical device (IMD),
wherein the IMD comprises at least a portion of the sensing circuitry and the processing circuitry, and
the method further comprising:
detecting, by the IMD, the precursor to the ventricular tachyarrhythmia; and
delivering, by the IMD, the non-shock therapy.

18. The method of claim 16, comprising:
a first medical device configured to apply the non-shock therapy to inhibit the onset of ventricular tachyarrhythmia;
a second device comprising at least a portion of:
the sensing circuitry configured to sense cardiac activity of a patient;
communication circuitry of the medical system, wherein the communication circuitry is configured to communicate between the first device and the second device; and
the processing circuitry, the method further comprising:
based on the sensed cardiac activity, detecting, by processing circuitry of the second device, a precursor to a ventricular tachyarrhythmia; and
responsive to detecting the precursor to the ventricular tachyarrhythmia, causing, by processing circuitry of the second device, a communication to the first device, the communication configured to trigger the first device to apply the non-shock therapy to inhibit the onset of the ventricular tachyarrhythmia.

19. The method of claim 18, wherein the first medical device is an external computing device,
wherein the non-shock therapy from the external computing device is configured to alert the patient via a user interface, and
wherein the alert comprises one or more of:
a recommendation to increase in oral antiarrhythmics for a predetermined duration,
a notice for an immediate self-injection of an antiarrhythmic substance including: adenosine, atropine, or lidocaine,
a recommendation to cease stressful activities or exercise,
a recommendation to cease caffeine intake, or
a notice to put on a wearable cardiac defibrillator.

20. A non-transitory computer-readable storage medium comprising instructions that, when executed, cause processing circuitry of a computing device to:
receive an indication of cardiac activity of a patient from sensing circuitry of a medical system, wherein:
the sensing circuitry is configured to sense cardiac activity of the patient, and
the computing device is a component of the medical system;
based on the sensed cardiac activity, detect a precursor to a ventricular tachyarrhythmia; and
responsive to detecting the precursor to the ventricular tachyarrhythmia, deliver a non-shock therapy to inhibit an onset of the ventricular tachyarrhythmia.
